(19) United States
(12) Patent Application Publication
(10) Pub. No.: US 2010/0016331 A1
(43) Pub. Date: Jan. 21, 2010

(54) NOVEL COMPOUNDS

(76) Inventors: Keith Biggadike, Hertfordshire (GB); Anthony William James Cooper, Hertfordshire (GB); David House, Hertfordshire (GB); Iain McFarlane McLay, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/444,719

(22) PCT Filed: Oct. 10, 2007
(86) PCT No.: PCT/EP2007/060776

§ 371 Date: Apr. 8, 2009

(30) Foreign Application Priority Data
Oct. 13, 2006 (GB) 0620406.9

(51) Int. Cl. A61K 31/497; C07D 231/56; A61K 31/415; C07D 403/08; C07D 401/08; A61K 31/445

(52) U.S. Cl. 514/254.06; 548/361.1; 514/403; 544/371; 546/199; 514/322

(57) ABSTRACT

The present invention provides compounds of formula (I):

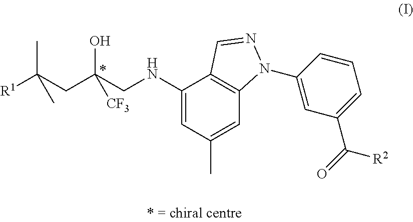

a process for their preparation, pharmaceutical compositions comprising the compounds and the preparation of said compositions, intermediates and use of the compounds for the manufacture of a medicament for therapeutic treatment, particularly for the treatment of inflammation, allergy and/or skin disease.

The present invention relates to non-steroidal compounds, pharmaceutical compositions comprising the compounds and the use of the compounds for the manufacture of a medicament, particularly for the treatment of inflammation and/or allergic conditions.

Nuclear receptors are a class of structurally related proteins involved in the regulation of gene expression. The steroid hormone receptors are a subset of this family whose natural ligands typically comprise endogenous steroids such as estradiol (estrogen receptor), progesterone (progesterone receptor) and cortisol (glucocorticoid receptor). Man-made ligands to these receptors play an important role in human health, in particular the use of glucocorticoid agonists to treat a wide range of inflammatory conditions.

Glucocorticoids exert their actions at the glucocorticoid receptor (GR) through at least two intracellular mechanisms, transactivation and transrepression (see: Schacke, H., Docke, W-D. & Asadullah, K. (2002) Pharmacol and Therapeutics 96:23-43; Ray, A., Siegel, M. D., Prefontaine, K. E. & Ray, P. (1995) Chest 107:139S; and Konig, H., Ponta, H., Rahmsdorf, H. J. & Herrlich, P. (1992) EMBO J 11:2241-2246). Transactivation involves direct binding of the glucocorticoid receptor to distinct deoxyribonucleic acid (DNA) glucocorticoid response elements (GREs) within gene promoters, usually but not always increasing the transcription of the downstream gene product. Recently, it has been shown that the GR can also regulate gene expression through an additional pathway (transrepression) in which the GR does not bind directly to DNA. This mechanism involves interaction of the GR with other transcription factors, in particular NFkB and AP1, leading to inhibition of their pro-transcriptional activity (Schacke, H., Docke, W-D. & Asadullah, K. (2002) Pharmacol and Therapeutics 96:23-43; and Ray, A., Siegel, M. D., Prefontaine, K. E. & Ray, P. (1995) Chest 107:139S). Many of the genes involved in the inflammatory response are transcriptionally activated through the NFkB and AP1 pathways and therefore inhibition of this pathway by glucocorticoids may explain their anti-inflammatory effect (see: Barnes, P. J. & Adcock, I. (1993) Trend Pharmacol Sci 14:436-441; and Cato, A. C. & Wade, E. (1996) Bioessays 18: 371-378).

Despite the effectiveness of glucocorticoids in treating a wide range of conditions, a number of side-effects are associated with pathological increases in endogenous cortisol or the use of exogenous, and particularly systemically administered, glucocorticoids. These include reduction in bone mineral density (Wong, C. A., Walsh, L. J., Smith, C. J. et al. (2000) Lancet 355:1399-1403), slowing of growth (Allen, D. B. (2000) Allergy 55: suppl 62, 15-18), skin bruising (Pauwels, R. A., Lofdahl, C. G., Latinen, L. A. et al. (1999) N Engl J Med 340:1948-1953), development of cataracts (Cumming, R. G., Mitchell, P. & Leeder, S. R. (1997) N Engl J Med 337:8-14) and dysregulation of lipid and glucose metabolism (Faul, J. L., Tormey, W., Tormey, V. & Burke, C. (1998) BMJ 317:1491; and Andrews, R. C. & Walker, B. R. (1999) Clin Sci 96:513-523). The side-effects are serious enough often to limit the dose of glucocorticoid that can be used to treat the underlying pathology leading to reduced efficacy of treatment.

Current known glucocorticoids have proved useful in the treatment of inflammation, tissue rejection, auto-immunity, various malignancies, such as leukemias and lymphomas, Cushing's syndrome, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia and Little's syndrome.

Glucocorticoids are especially useful in disease states involving systemic inflammation such as inflammatory bowel disease, systemic lupus erythematosus, polyarteritis nodosa, Wegener's granulomatosis, giant cell arteritis, rheumatoid arthritis, osteoarthritis, seasonal rhinitis, allergic rhinitis, vasomotor rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis and cirrhosis. Glucocorticoids have also been used as immunostimulants and repressors and as wound healing and tissue repair agents.

Glucocorticoids have also found use in the treatment of diseases such as inflammatory scalp alopecia, panniculitis, psoriasis, discoid lupus erythemnatosus, inflamed cysts, atopic dermatitis, pyoderma gangrenosum, pemphigus vulgaris, bullous pemphigoid, systemic lu pus erythematosus, dermatomyositis, herpes gestation is, eosinoph ilic fasciitis, relapsing polychondritis, inflammatory vasculitis, sarcoidosis, Sweet's disease, type 1 reactive leprosy, capillary hemangiomas, contact dermatitis, atopic dermatitis, lichen planus, exfoliative dermatitus, erythema nodosum, acne, hirsutism, toxic epidermal necrolysis, erythema multiform and cutaneous T-cell lymphoma.

In one embodiment, the present invention provides compounds of formula (I):

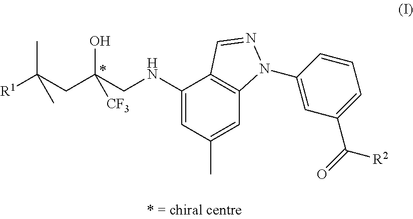

wherein

R¹ represents 5-fluoro-2-methoxy-phenyl or 5-fluoro-2-hydroxy-phenyl;

R² represents —NR³R⁴;

R³ represents hydrogen, and

R⁴ represents hydrogen, —CH₂CH₃, —CH₂CH(CH₃)₂, —CH₂CH₂OH, —CH₂CH(OH)CH₃, —CH₂CH(OH)CH₂CH₃, —CH₂CH(OH)CH₂OH, —CH(CONH₂)CH₂CH₃, —CH₂CH₂CONH₂, CH(CONH₂)CH₂CH₂CONH₂, —CH(CONH₂)CH(OH)CH₃, —CH(CONH₂)CH₂CONH₂, —CH(CONH₂)CH(CH₃)₂, —CH(CONH₂)CH₂CH₂SCH₃, cyclopropyl, cyclopentyl, 2-oxotetrahydro-3-furanyl, 3-furanyl-methyl, 1,1-dioxidotetrahydro-3-thienyl, 1,2,4-oxadiazol-3-ylmethyl, (3-methyl-1H-1,2,4-triazol-5-yl)methyl, 1 -methyl-2-oxo-3-pyrrolidinyl, 5-oxo-3-pyrrolidinyl, 2-amino-2-oxo-1-phenylethyl, 1-(aminocarbonyl)cyclopropyl or 1-(aminocarbonyl)cyclobutyl; or

R³ and R⁴ together with the nitrogen atom to which they are attached, form

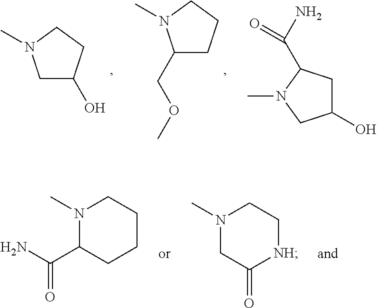

salts thereof (hereinafter “compounds of the invention”).

In a further embodiment, the present invention provides compounds of formula (IA):

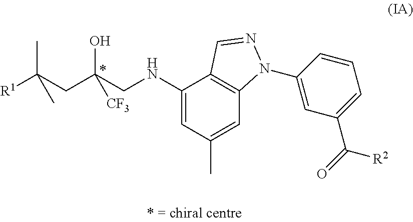

wherein

R¹ represents 5-fluoro-2-methoxy-phenyl or 5-fluoro-2-hydroxy-phenyl;

R² represents —NR³R⁴;

R³ represents hydrogen, and

R⁴ represents hydrogen, —CH₂CH₃, —CH₂CH(CH₃)₂, —CH₂CH₂0H, —CH₂CH(OH)CH₃, —CH₂CH(OH)CH₂CH₃, —CH₂CH(OH)CH₂OH, —CH(CONH₂)CH₂CH₃, —CH₂CH₂CONH₂CH(CONH₂)CH₂CH₂CONH₂, -CH(CONH₂)CH(OH)CH₃, —CH(CONH₂)CH₂CONH₂, —CH(CONH₂)CH(CH₃)₂, —CH(CONH₂)CH₂CH₂SCH₃, cyclopropyl, cyclopentyl, 2-oxotetrahydro-3-furanyl, 3-furanyl-methyl, 1,1-dioxidotetrahydro-3-thienyl, 1,2,4-oxadiazol-3-ylmethyl or (3-methyl-1H-1,2,4-triazol-5-yl)methyl; or

R³ and R⁴ together with the nitrogen atom to which they are attached, form

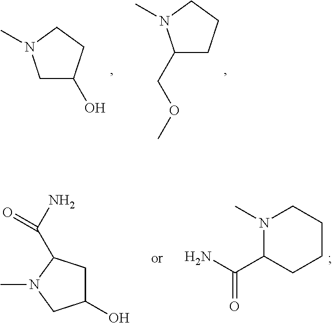

and salts thereof.

In one embodiment, R¹ represents 5-fluoro-2-methoxy-phenyl. In a further embodiment, R¹ represents 5-fluoro-2-hydroxy-phenyl.

In one embodiment, R³ represents hydrogen.

In one embodiment, R³ represents hydrogen and R⁴ represents hydrogen, —CH₂CH₃, —CH₂CH(CH₃)₂, —CH₂CH₂OH, —CH₂CH(OH)CH₃, —CH₂CH(OH)CH₂CH₃, —CH₂CH(OH)CH₂OH, —CH(CONH₂)CH₂CH₃, —CH₂CH₂CONH₂, —CH(CONH₂)CH₂CH₂—CH(CONH₂)CH(OH)CH₃, —CH(CONH₂)CH₂CONH₂, —CH(CONH₂)CH(CH₃)₂, —CH(CONH₂)CH₂CH₂SCH₃, cyclopropyl, cyclopentyl, 2-oxotetrahydro-3-furanyl, 3-furanyl-methyl, 1,1-dioxidotetrahydro-3-thienyl, 1,2,4-oxadiazol-3-ylmethyl or (3-methyl-1H-1,2,4-triazol-5-yl)methyl; or

R³ and R⁴ together with the nitrogen atom to which they are attached, form

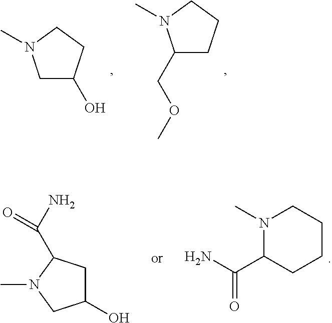

In one embodiment, R⁴ represents —CH₂CH₃, —CH₂CH₂OH, —CH₂CH(OH)CH₃, —CH(CONH₂)CH₂CH₃, —CH₂CH₂CONH₂, —CH(CONH₂)CH₂CH₂CONH₂, CH(CONH₂)CH(CH₃)₂, cyclopropyl or 1,1-dioxidotetrahydro-3-thienyl. In another embodiment, R⁴ represents —CH₂CH₃, —CH₂CH₂OH, —CH₂CH(OH)CH₃, or 1,1-dioxidotetrahydro-3-thienyl. In another embodiment R⁴ represents 5-oxo-3-pyrrolidinyl

It is to be understood that the present invention encompasses all combinations of the substituent groups described above.

In one embodiment, the compound of formula (I) is:

N-ethyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(2-methylpropyl)benzamide;

N-cyclopropyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-cyclopentyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(2-hydroxyethyl)benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(2S)-2-hydroxypropyl]benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(2R)-2-hydroxypropyl]benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(2-hydroxybutyl)benzamide;

N-[(2S)-2,4-dihydroxybutyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-[(2R)-2,4-dihydroxybutyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-[(1S)-1-(aminocarbonyl)propyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-(3-amino-3-oxopropyl)-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N²-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-D-glutamamide;

N-[(1S,2R)-1-(aminocarbonyl)-2-hydroxypropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N²-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-L-aspartamide;

N-[(1S)-1-(aminocarbonyl)-3-(methylthio)propyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-[(1S)-1-(aminocarbonyl)-2-methylpropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-[(1R)-1-(aminocarbonyl)-2-methylpropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

1-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-2-piperidinecarboxamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(3R)-2-oxotetrahydro-3-furanyl]benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(3S)-2-oxotetrahydro-3-furanyl]benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(3S)-2-oxotetrahydro-3-furanyl]benzamide Enantiomer 1;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(3S)-2-oxotetrahydro-3-furanyl]benzamide Enantiomer 2;

1,1,1-trifluoro-4-[5-fluoro-2-(methyloxy)phenyl]-4-methyl-2-[({6-methyl-1-[3-({(2S)-2-[(methyloxy)methyl]-1-pyrrolidinyl}carbonyl)phenyl]-1H-indazol-4-yl}amino)methyl]-2-pentanol;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(3-furanylmethyl)benzamide;

N-(1,1-dioxidotetrahydro-3-thienyl)-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

(3R)-1-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-3-pyrrolidinol;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(1,2,4-oxadiazol-3-ylmethyl)benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(3-methyl-1H-1,2,4-triazol-yl)methyl]benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

(4S)-1-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-4-hydroxy-D-prolinamide;

(4R)-1-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-4-hydroxy-D-prolinamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(3R)-1-methyl-2-oxo-3-pyrrolidinyl]benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(5-oxo-3-pyrrolidinyl)benzamide;

4-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-2-piperazinone;

N-[(1R)-2-amino-2-oxo-1-phenylethyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-[1-(aminocarbonyl)cyclopropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-[1-(aminocarbonyl)cyclobutyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide; or a salt thereof.

In another embodiment, the compound of formula (I) is:

N-ethyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(2-methylpropyl)benzamide;

N-cyclopropyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-cyclopentyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(2-hydroxyethyl)benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(2S)-2-hydroxypropyl]benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(2R)-2-hydroxypropyl]benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(2-hydroxybutyl)benzamide;

N-[(2S)-2,4-dihydroxybutyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-[(2R)-2,4-dihydroxybutyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-[(1S)-1-(aminocarbonyl)propyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-(3-amino-3-oxopropyl)-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N²-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-D-glutamamide;

N-[(1S,2R)-1-(aminocarbonyl)-2-hydroxypropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N²-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-L-aspartamide;

N-[(1S)-1-(aminocarbonyl)-3-(methylthio)propyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-[(1S)-1-(aminocarbonyl)-2-methylpropyl]-3-(4-[4-[5-fluoro-2-(methyloxy)phenyl]-2-hydroxy-4-methyl-2-(trifluoromethyl)pentyl]amino}-6-methyl-1H-indazol-1-yl)benzamide;

N-[(1R)-1-(aminocarbonyl)-2-methylpropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

1-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-2-piperidinecarboxamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(3R)-2-oxotetrahydro-3-furanyl]benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(3S)-2-oxotetrahydro-3-furanyl]benzamide;

1,1,1-trifluoro-4-[5-fluoro-2-(methyloxy)phenyl]-4-methyl-2-[({6-methyl-1-[3-({(2S)-2-[(methyloxy)methyl]-1-pyrrolidinyl}carbonyl)phenyl]-1H-indazol-4-yl}amino)methyl]-2-pentanol;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(3-furanylmethyl)benzamide;

N-(1,1-dioxidotetrahydro-3-thienyl)-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

(3R)-1-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-3-pyrrolidinol;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(1,2,4-oxadiazol-3-ylmethyl)benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(3-methyl-1H-1,2,4-triazol-5-yl)methyl]benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

(4S)-1-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-4-hydroxy-D-prolinamide;

(4R)-1-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-4-hydroxy-D-prolinamide; or

a salt thereof.

In another embodiment, the compound of formula (I) is:

N-ethyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-cyclopropyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(2-hydroxyethyl)benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(2R)-2-hydroxypropyl]benzamide;

N-[(1S)-1-(aminocarbonyl)propyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-(3-amino-3-oxopropyl)-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N²-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-D-glutamamide;

N-[(1R)-1-(aminocarbonyl)-2-methylpropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-(1 1-dioxidotetrahydro-3-thienyl)-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-(5-oxo-3-pyrrolidinyl)benzamide; or

a salt thereof.

In a further embodiment, the compound of formula (I) is:

N-ethyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-cyclopropyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide; 3 3-(4-{[4-[5-fluoro-2-(methyloxy)phenyl]-2-hydroxy-4-methyl-2-(trifluoromethyl)pentyl]amino}-6-methyl-1H-indazol-1-yl)-N-(2-hydroxyethyl)benzamide;

3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(2R)-2-hydroxypropyl]benzamide;

N-[(1S)-1-(aminocarbonyl)propyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-(3-amino-3-oxopropyl)-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N²-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-D-glutamamide;

N-[(1R)-1-(aminocarbonyl)-2-methylpropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;

N-(1,1-dioxidotetrahydro-3-thienyl)-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide; or

a salt thereof.

The compounds of the invention may provide agonism of the glucocorticoid receptor.

The compounds of formula (I) each contain one or more chiral centres so there are at least two possible stereoisomers (enantiomers or diastereomers) of each compound of formula (I). Further, at least one of the possible enantiomers or diastereomers of each compound of formula (I) modulates the glucocorticoid receptor.

The terms diastereomer A and diastereomer B are used herein to refer to the diastereomers of a compound of formula (I) based on their order of elution using the chiral chromatographic methodology described herein. Diastereomer A refers to the first diastereomer to elute, and diastereomer B refers to the second diastereomer to elute.

It will be appreciated by those skilled in the art that although the absolute retention time on chromatography can be variable, the order of elution remains the same when the same column and conditions are employed. However, the use of a different chromatography column and conditions may alter the order of elution.

It will be appreciated by those skilled in the art that at least one isomer (e.g. one enantiomer of a racemate or one diastereomer) has the described activity. The other isomers may have similar activity, less activity, no activity or may have some antagonist activity in a functional assay.

One embodiment of the invention encompasses a compound of the invention in the form of a single enantiomer or diastereomer or mixture of isomers (e.g. racemic mixture). Thus in one embodiment the compound of the invention is in the form of a diastereomer or a mixture of diastereomers. In another embodiment of the invention, the compound of the invention is diastereomer A. In a further embodiment of the invention, the compound of the invention is diastereomer B.

Included within the scope of the “compounds of the invention” are all solvates (including hydrates), complexes, polymorphs, prodrugs, radiolabelled derivatives, stereoisomers and optical isomers of the compounds of formula (I) and salts thereof.

The compounds of the invention may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The skilled artisan will further appreciate that certain compounds of the invention that exist in crystalline form, including the various solvates thereof, may exhibit polymorphism (i.e. the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

One embodiment of the invention embraces compounds of formula (I) and salts and solvates thereof. Another embodiment of the invention embraces compounds of formula (I) and salts thereof. Another embodiment of the invention embraces compounds of formula (I) and solvates thereof. A further embodiment of the invention embraces compounds of formula (I) as the free base.

Salts and solvates of the compounds of formula (I) which are suitable for use in medicine are those wherein the counter-ion or associated solvent is pharmaceutically acceptable. However, salts and solvates having non-pharmaceutically acceptable counter-ions or associated solvents are within the scope of the present invention, for example, for use as intermediates in the preparation of other compounds of formula (I) and their pharmaceutically acceptable salts and solvates.

Suitable salts according to the invention include those formed with both organic and inorganic acids or bases. Pharmaceutically acceptable acid addition salts include those formed from hydrochloric, hydrobromic, sulphuric, citric, tartaric, phosphoric, lactic, pyruvic, acetic, trifluoroacetic, triphenylacetic, sulphamic, sulphanilic, succinic, oxalic, fumaric, maleic, malic, glutamic, aspartic, oxaloacetic, methanesulphonic, ethanesulphonic, arylsulphonic (for example p-toluenesulphonic, benzenesulphonic, naphthalenesulphonic or naphthalenedisulphonic), salicylic, glutaric, gluconic, tricarballylic, cinnamic, substituted cinnamic (for example, phenyl, methyl, methoxy or halo substituted cinnamic, including 4-methyl and 4-methoxycinnamic acid), ascorbic, oleic, naphthoic, hydroxynaphthoic (for example 1- or 3-hydroxy-2-naphthoic), naphthaleneacrylic (for example naphthalene-2-acrylic), benzoic, 4-methoxybenzoic, 2- or 4-hydroxybenzoic, 4-chlorobenzoic, 4-phenylbenzoic, benzeneacrylic (for example 1,4-benzenediacrylic) and isethionic acids. Pharmaceutically acceptable base salts include ammonium salts, for example those formed with pharmaceutically acceptable organic primary, secondary, and tertiary amines including aliphatic amines, aromatic amines, aliphatic diamines, and hydroxy alkylamines such as methylamine, ethylamine, 2-hydroxyethylamine, diethylamine, TEA, ethylenediamine, ethanolamine, diethanolamine, and cyclohexylamine; alkali metal salts such as those of lithium, sodium and potassium, alkaline earth metal salts such as those of calcium and magnesium, aluminum, and zinc salts; carbonates and bicarbonates of a pharmaceutically acceptable metal cation such as sodium, potassium, lithium, calcium, magnesium, aluminum, and zinc; and salts with organic bases such as dicyclohexylamine and N-methyl-D-glucamine.

The compounds of formula (I) and pharmaceutically acceptable salts thereof are expected to have potentially beneficial anti-inflammatory or anti-allergic effects, particularly upon topical administration, demonstrated by, for example, their ability to bind to the glucocorticoid receptor and to illicit a response via that receptor. Hence, the compounds of formula (I) and pharmaceutically acceptable salts thereof may be of use in the treatment of inflammatory and/or allergic disorders.

Examples of disease states in which the compounds of formula (I) and pharmaceutically acceptable salts thereof are expected to have utility include skin diseases such as eczema, psoriasis, allergic dermatitis, neurodermatitis, pruritis and hypersensitivity reactions; inflammatory conditions of the nose, throat or lungs such as asthma (including allergen-induced asthmatic reactions), rhinitis (including hayfever), nasal polyps, chronic obstructive pulmonary disease (COPD), interstitial lung disease, and fibrosis; inflammatory bowel conditions such as ulcerative colitis and Crohn's disease; and auto-immune diseases such as rheumatoid arthritis.

The term “rhinitis” is used herein to refer to all types of rhinitis including allergic rhinitis such as seasonal rhinitis (for example hayfever) or perennial rhinitis, and non-allergic rhinitis or vasomotor rhinitis.

It will be appreciated by those skilled in the art that reference herein to treatment extends to prophylaxis as well as the treatment of established conditions.

As mentioned above, compounds of formula (I) and pharmaceutically acceptable salts thereof are expected to be of use in human or veterinary medicine, in particular as anti-inflammatory and/or anti-allergic agents.

There is thus provided as a further aspect of the invention a compound of formula (I) or a pharmaceutically acceptable salt thereof for use in human or veterinary medicine, particularly in the treatment of patients with inflammatory and/or allergic conditions, such as rheumatoid arthritis, asthma, COPD, allergy and/or rhinitis.

There is further provided as a further aspect of the invention a compound of formula (I) or a pharmaceutically acceptable salt thereof for use in the treatment of patients with rhinitis.

Further provided is a compound of formula (I) or a pharmaceutically acceptable salt thereof for use in the treatment of patients with skin disease such as eczema, psoriasis, allergic dermatitis, neurodermatitis, pruritis and/or hypersensitivity reactions.

According to another aspect of the invention, there is provided the use of a compound of formula (I) or a pharmaceutically acceptable salt thereof for the manufacture of a medicament for the treatment of patients with inflammatory and/or allergic conditions, such as rheumatoid arthritis, asthma, COPD, allergy and/or rhinitis.

According to another aspect of the invention, there is provided the use of a compound of formula (I) or a pharmaceutically acceptable salt thereof for the manufacture of a medicament for the treatment of patients with rhinitis.

According to yet to another aspect of the invention, there is provided the use of a compound of formula (I) or a pharmaceutically acceptable salt thereof for the manufacture of a medicament for the treatment of patients with skin disease such as eczema, psoriasis, allergic dermatitis, neurodermatitis, pruritis and/or hypersensitivity reactions.

In a further or alternative aspect, there is provided a method for the treatment of a human or animal subject with an inflammatory and/or allergic condition such as rheumatoid arthritis, asthma, COPD, allergy and/or rhinitis, which method comprises administering to said human or animal subject an effective amount of a compound of formula (I) or a pharmaceutically acceptable salt thereof.

In a further or alternative aspect, there is provided a method for the treatment of a human or animal subject with rhinitis, which method comprises administering to said human or animal subject an effective amount of a compound of formula (I) or a pharmaceutically acceptable salt thereof.

In yet a further or alternative aspect, there is provided a method for the treatment of a human or animal subject with skin disease such as eczema, psoriasis, allergic dermatitis, neurodermatitis, pruritis and/or hypersensitivity reactions, which method comprises administering to said human or animal subject an effective amount of a compound of formula (I) or a pharmaceutically acceptable salt thereof.

The compounds of formula (I) and pharmaceutically acceptable salts thereof may be formulated for administration in any convenient way, and the invention therefore also includes within its scope pharmaceutical compositions comprising a compound of formula (I) or a pharmaceutically acceptable salt thereof together, if desirable, in admixture with one or more physiologically acceptable diluents or carriers.

Further, there is provided a process for the preparation of such pharmaceutical compositions which comprises mixing the ingredients. A pharmaceutical composition comprising a compound of formula (I) or a pharmaceutically acceptable salt thereof may be prepared by, for example, admixture at ambient temperature and atmospheric pressure.

Pharmaceutical compositions comprising a compound of formula (I) or a pharmaceutically acceptable salt thereof may be suitable for topical administration (which includes epicutaneous, inhaled, intranasal or ocular administration), enteral administration (which includes oral or rectal administration) or parenteral administration (such as by injection or infusion). The compounds of formula (I) and pharmaceutically acceptable salts thereof may, for example, be formulated for oral, buccal, sublingual, parenteral, local rectal administration or other local administration.

Pharmaceutical compositions may be in the form of, for example, solutions or suspensions (aqueous or non-aqueous), tablet, capsules, oral liquid preparations, powders, granules, lozenges, lotions, creams, ointments, gels, foams, reconstitutable powders or suppositories as required by the route of administration.

Generally, compositions containing a compound of formula (I) or a pharmaceutically acceptable salt thereof may contain from about 0.1 to about 99%, such as from about 10 to about 60%, by weight based on the total weight of the composition, of the compound of formula (I) or a pharmaceutically acceptable salt thereof, depending on the route of administration. The dose of the compound used in the treatment of the abovementioned disorders will vary in the usual way with the seriousness of the disorders, the weight of the sufferer and other similar factors. However, as a general guide, suitable unit does may be about 0.001 to about 100mg, for example about 0.001 to about 1mg, and such unit doses may be administered more than once a day, for example two or three times a day. Such therapy may extend for a number of weeks or month.

The compounds of formula (I) and pharmaceutically acceptable salts thereof may, for example, be formulated for oral, buccal, sublingual, parenteral, local rectal administration or other local administration.

Local administration as used herein includes administration by insufflation and inhalation. Examples of various types of preparation for local administration include ointments, lotions, creams, gels, foams, preparations for delivery by transdermal patches, powders, sprays, aerosols, capsules or cartridges for use in an inhaler or insufflator or drops (e.g. eye or nose drops), solutions/suspensions for nebulisation, suppositories, pessaries, retention enemas and chewable or suckable tablets or pellets (e.g. for the treatment of aphthous ulcers) or liposome or microencapsulation preparations.

The proportion of the active compound of formula (I) or a pharmaceutically acceptable salt thereof in the local compositions according to the invention depends on the precise type of composition to be prepared, and the route of administration, but will generally be within the range of from 0.001 to 10% by weight based on the total weight of the composition. Generally, for most types of preparations, the proportion used will be within the range of from 0.005 to 1%, for example from 0.01 to 1%, such as 0.01 to 0.5% by weight based on the total weight of the composition. However, in powders for inhalation or insufflation the proportion used will normally be within the range of from 0.1 to 5% by weight based on the total weight of the composition.

In one embodiment, pharmaceutical compositions comprising a compound of formula (I) or a pharmaceutically acceptable salt thereof may be suitable for topical administration, for example for intranasal or inhaled administration. Inhaled administration involves topical administration to the lung, such as by aerosol or dry powder composition.

Generally, compositions suitable for intranasal or inhaled administration may conveniently be formulated as aerosols, solutions, suspensions, drops, gels or dry powders, optionally with one or more physiologically acceptable diluents and/or carriers such as aqueous or non-aqueous vehicles, thickening agents, isotonicity adjusting agents, antioxidants and/or preservatives.

For compositions suitable for intranasal or inhaled administration, the compound of formula (I) or a pharmaceutically acceptable salt thereof may be in a particle-size-reduced form prepared by, for example, micronisation and milling. Generally, the size-reduced (e.g. micronised) compound can be defined by a D₅₀ value of about 0.5 to about 10 microns (for example as measured using laser diffraction).

In one embodiment, pharmaceutical compositions comprising a compound of formula (I) or a pharmaceutically acceptable salt thereof are suitable for intranasal administration. For example, the compounds of formula (I) or a pharmaceutically acceptable salt thereof may be formulated for intranasal use in man either as a solution composition or a suspension composition, for example as a solution composition such as an aqueous solution composition.

A suitable dosing regime for an intranasal composition may be for the patient to inhale slowly through the nose subsequent to the nasal cavity being cleared. During inhalation, the composition may be administered to one nostril while the other is manually compressed. This procedure may then be repeated for the other nostril. Generally, one or two sprays per nostril may be administered by the above procedure up to two or three times each day. In one embodiment, the intranasal compositions comprising a compound of formula (I) or a pharmaceutically acceptable salt thereof are suitable for once daily administration. Typically, each spray to the nostril may deliver from about 25 to about 100 μL of intranasal composition. Further, generally, each spray to the nostril may deliver from about 1 to about 100 μg, for example about 1 to about 50 μg, of the compound of formula (I) or a pharmaceutically acceptable salt thereof.

Intranasal compositions comprising a compound of formula (I) or a pharmaceutically acceptable salt thereof may permit the compound to be delivered to all areas of the nasal cavities (the target tissue) and further, may permit the compound to remain in contact with the target tissue for longer periods of time. Compositions comprising a compound of formula (I) or a pharmaceutically acceptable salt thereof, suitable for intranasal administration, may optionally contain one or more suspending agents, one or more preservatives, one or more wetting agents and/or one or more isotonicity adjusting agents as desired. Compositions suitable for intranasal administration may optionally further contain other excipients such as antioxidants (for example sodium metabisulphite), taste-masking agents (for example menthol) and sweetening agents (for example dextrose, glycerol, saccharin and/or sorbitol). Excipients that may be employed in intranasal compositions include, for example, xylitol, potassium sorbate, EDTA, sodium citrate, citric acid, polysorbate 80 and Avicel CL611.

The suspending agent, if included, will typically be present in the intranasal composition in an amount of between about 0.1 and 5%, such as between about 1.5 and 2.4%, by weight based on the total weight of the composition. Examples of suspending agents include Avicel, carboxymethylcellulose, veegum, tragacanth, bentonite, methylcellulose and polyethylene glycols, e.g. microcrystalline cellulose or carboxy methylcellulose sodium. Suspending agents may also be included in, for example, compositions suitable for inhaled, ocular and oral administration, as appropriate.

For stability purposes, intranasal compositions comprising a compound of formula (I) or a pharmaceutically acceptable salt thereof may be protected from microbial or fungal contamination and growth by inclusion of a preservative. Examples of pharmaceutically acceptable anti-microbial agents or preservatives may include quaternary ammonium compounds (e.g. benzalkonium chloride, benzethonium chloride, cetrimide and cetylpyridinium chloride), mercurial agents (e.g. phenylmercuric nitrate, phenylmercuric acetate and thimerosal), alcoholic agents (e.g. chlorobutanol, phenylethyl alcohol and benzyl alcohol), antibacterial esters (e.g. esters of para-hydroxybenzoic acid), chelating agents such as disodium edetate (EDTA) and other anti-microbial agents such as chlorhexidine, chlorocresol, sorbic acid and its salts (such as potassium sorbate) and polymyxin. Examples of pharmaceutically acceptable anti-fungal agents or preservatives may include sodium benzoate. In one embodiment, there is provided a pharmaceutical composition comprising a compound of formula (I) or a pharmaceutically acceptable salt thereof which is benzalkonium chloride-free. The preservative, if included, may be present in an amount of between about 0.001 and about 1%, such as about 0.015%, by weight based on the total weight of the composition. Preservatives may be included in composition suitable for other routes of administration as appropriate.

Compositions which contain a suspended medicament may include a pharmaceutically acceptable wetting agent which functions to wet the particles of the medicament to facilitate dispersion thereof in the aqueous phase of the composition. Typically, the amount of wetting agent used will not cause foaming of the dispersion during mixing. Examples of wetting agents include fatty alcohols, esters and ethers, such as polyoxyethylene (20) sorbitan monooleate (polysorbate 80). The wetting agent may be present in the composition in an amount of between about 0.001 and about 1%, for example between about 0.005% and about 1%, by weight based on the total weight of the composition. Wetting agents may be included in compositions suitable for other routes of administration, e.g. for inhaled or ocular administration, as appropriate.

An isotonicity adjusting agent may be included to achieve isotonicity with body fluids e.g. fluids of the nasal cavity, resulting in reduced levels of irritancy. Examples of isotonicity adjusting agents include sodium chloride, dextrose, xylitol and calcium chloride. An isotonicity agent may be included in the composition in an amount of between about 0.1 and 10%, such as about 4.5% by weight based on the total weight of the composition. Isotonicity adjusting agents may also be included in, for example, compositions suitable for inhaled, ocular, oral and parenteral forms of administration, as appropriate.

Further, intranasal compositions may be buffered by the addition of suitable buffering agents such as sodium citrate, citric acid, phosphates such as disodium phosphate (for example dodecahydrate, heptahydrate, dihydrate and anhydrous forms) or sodium phosphate and mixtures thereof. Buffering agents may also be included in compositions suitable for other routes of administration, as appropriate.

Compositions for administration topically to the nose for example, for the treatment of rhinitis, include pressurised aerosol compositions and aqueous compositions administered to the nose by pressurised pump. In one embodiment, the present invention encompasses compositions which are non-pressurised and adapted to be administered topically to the nasal cavity. Suitable compositions contain water as the diluent or carrier for this purpose. Aqueous compositions for administration to the lung or nose may be provided with conventional excipients such as buffering agents, tonicity modifying agents and the like. Aqueous compositions may also be administered to the nose by nebulisation.

The compounds of formula (I) and pharmaceutically acceptable salts thereof may be formulated as a fluid composition for delivery from a fluid dispenser, for example a fluid dispenser having a dispensing nozzle or dispensing orifice through which a metered dose of the fluid composition is dispensed upon the application of a user-applied force to a pump mechanism of the fluid dispenser. Such fluid dispensers are generally provided with a reservoir of multiple metered doses of the fluid composition, the doses being dispensable upon sequential pump actuations. The dispensing nozzle or orifice may be configured for insertion into the nostrils of the user for spray dispensing of the fluid composition into the nasal cavity. A fluid dispenser of the aforementioned type is described and illustrated in WO05/044354, the entire content of which is hereby incorporated herein by reference. The dispenser has a housing which houses a fluid discharge device having a compression pump mounted on a container for containing a fluid compositions. The housing has at least one finger-operable side lever which is movable inwardly with respect to the housing to cam the container upwardly in the housing to cause the pump to compress and pump a metered dose of the composition out of a pump stem through a nasal nozzle of the housing. In one embodiment, the fluid dispenser is of the general type illustrated in FIGS. 30-40 of WO05/044354.

Spray compositions may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain a compound of formula (I) or a pharmaceutically acceptable salt thereof and a suitable propellant such as a fluorocarbon or hydrogen-containing chlorofluorocarbon or mixtures thereof, particularly hydrofluoroalkanes, especially 1,1,1,2-tetrafluoroethane, 1,1,1,2,3,3,3-heptafluoro-n-propane or a mixture thereof. The aerosol composition may optionally contain additional formulation excipients well known in the art such as surfactants e.g. oleic acid, lecithin or an oligolactic acid or derivative e.g. as described in WO94/21229 and WO98/34596 and cosolvents, for example ethanol.

There is thus provided as a further aspect of the invention a pharmaceutical aerosol formulation comprising a compound of formula (I) or a pharmaceutically acceptable salt thereof and a fluorocarbon or hydrogen-containing chlorofluorocarbon as propellant, optionally in combination with a surfactant and/or a cosolvent.

According to another aspect of the invention, there is provided a pharmaceutical aerosol formulation wherein the propellant is selected from 1,1,1,2-tetrafluoroethane, 1,1,1,2,3,3,3-heptafluoro-n-propane and mixtures thereof.

The formulations of the invention may be buffered by the addition of suitable buffering agents.

Aerosol compositions may be presented in single or multidose quantities in sterile form in a sealed container, which may take the form of a cartridge or refill for use with an atomising device or inhaler. Alternatively, the sealed container may be a unitary dispensing device such as a single dose nasal inhaler or an aerosol dispenser fitted with a metering valve (metered dose inhaler), which is intended for disposal once the contents of the container have been exhausted.

Capsules and cartridges for use in an inhaler or insufflator, of for example gelatine, may be formulated containing a powder mix for inhalation of a compound of formula (I) or a pharmaceutically acceptable salt thereof and a suitable powder base such as lactose or starch. Each capsule or cartridge may generally contain from 20 μg to 10 mg of the compound of formula (I) or a pharmaceutically acceptable salt thereof. Alternatively, the compound of formula (I) or a pharmaceutically acceptable salt thereof may be presented without excipients such as lactose.

Optionally, in particular for dry powder inhalable compositions, a composition suitable for inhaled administration may be incorporated into a plurality of sealed dose containers (e.g. containing the dry powder composition) mounted longitudinally in a strip or ribbon inside a suitable inhalation device. The container is rupturable or peel-openable on demand and the dose of e.g. the dry powder composition may be administered by inhalation via a device such as the DISKUS™ device, marketed by GlaxoSmithKline. The DISKUS™ inhalation device is, for example, described in GB2242134A, and in such a device, at least one container for the composition in powder form (the container or containers preferably being a plurality of sealed dose containers mounted longitudinally in a strip or ribbon) is defined between two members peelably secured to one another; the device comprises: a means of defining an opening station for the said container or containers; a means for peeling the members apart at the opening station to open the container; and an outlet, communicating with the opened container, through which a user can inhale the composition in powder form from the opened container.

The proportion of the active compound of formula (I) or a pharmaceutically acceptable salt thereof in the local compositions according to the invention depends on the precise type of formulation to be prepared but will generally be within the range of from 0.001 to 10% by weight. Generally, for most types of preparations, the proportion used will be within the range of from 0.005 to 1%, for example from 0.01 to 0.5%. However, in powders for inhalation or insufflation the proportion used will normally be within the range of from 0.1 to 5%.

Aerosol formulations are preferably arranged so that each metered dose or “puff” of aerosol contains from 20 μg to 10 mg, preferably from 20 μg to 2000 μg, more preferably from 20 μg to 500 μg of a compound of formula (I) or a pharmaceutically acceptable salt thereof. Administration may be once daily or several times daily, for example 2, 3, 4 or 8 times, giving for example 1, 2 or 3 doses each time. The overall daily dose with an aerosol will be within the range from 100 μg to 10 mg, preferably from 200 μg to 2000 μg. The overall daily dose and the metered dose delivered by capsules and cartridges in an inhaler or insufflator will generally be double that delivered with aerosol formulations.

In the case of suspension aerosol formulations, the particle size of the particulate (e.g., micronised) drug should be such as to permit inhalation of substantially all the drug into the lungs upon administration of the aerosol formulation and will thus be less than 100 microns, desirably less than 20 microns, and in particular in the range of from 1 to 10 microns, such as from 1 to 5 microns, more preferably from 2 to 3 microns.

The formulations of a compound of formula (I) or a pharmaceutically acceptable salt thereof may be prepared by dispersal or dissolution of the medicament and a compound of formula (I) or a pharmaceutically acceptable salt thereof in the selected propellant in an appropriate container, for example, with the aid of sonication or a high-shear mixer. The process is desirably carried out under controlled humidity conditions.

The chemical and physical stability and the pharmaceutical acceptability of the aerosol formulations according to the invention may be determined by techniques well known to those skilled in the art. Thus, for example, the chemical stability of the components may be determined by HPLC assay, for example, after prolonged storage of the product. Physical stability data may be gained from other conventional analytical techniques such as, for example, by leak testing, by valve delivery assay (average shot weights per actuation), by dose reproducibility assay (active ingredient per actuation) and spray distribution analysis.

The stability of the suspension aerosol formulations according to the invention may be measured by conventional techniques, for example, by measuring flocculation size distribution using a back light scattering instrument or by measuring particle size distribution by cascade impaction or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to “fine particle fraction” which is the amount of active ingredient collected in the lower impingement chamber per actuation expressed as a percentage of the total amount of active ingredient delivered per actuation using the twin impinger method described above.

The term “metered dose inhaler” or MDI means a unit comprising a can, a secured cap covering the can and a formulation metering valve situated in the cap. MDI system includes a suitable channelling device. Suitable channelling devices comprise for example, a valve actuator and a cylindrical or cone-like passage through which medicament may be delivered from the filled canister via the metering valve to the nose or mouth of a patient such as a mouthpiece actuator.

MDI canisters generally comprise a container capable of withstanding the vapour pressure of the propellant used such as a plastic or plastic-coated glass bottle or preferably a metal can, for example, aluminium or an alloy thereof which may optionally be anodised, lacquer-coated and/or plastic-coated (for example incorporated herein by reference WO96/32099 wherein part or all of the internal surfaces are coated with one or more fluorocarbon polymers optionally in combination with one or more non-fluorocarbon polymers), which container is closed with a metering valve. The cap may be secured onto the can via ultrasonic welding, screw fitting or crimping. MDls taught herein may be prepared by methods of the art (eg., see Byron, above and WO96/32099). Preferably the canister is fitted with a cap assembly, wherein a drug-metering valve is situated in the cap, and said cap is crimped in place.

In one embodiment of the invention the metallic internal surface of the can is coated with a fluoropolymer, most preferably blended with a non-fluoropolymer. In another embodiment of the invention the metallic internal surface of the can is coated with a polymer blend of polytetrafluoroethylene (PTFE) and polyethersulfone (PES). In a further embodiment of the invention the whole of the metallic internal surface of the can is coated with a polymer blend of polytetrafluoroethylene (PTFE) and polyethersulfone (PES).

The metering valves are designed to deliver a metered amount of the formulation per actuation and incorporate a gasket to prevent leakage of propellant through the valve. The gasket may comprise any suitable elastomeric material such as, for example, low density polyethylene, chlorobutyl, bromobutyl, EPDM, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak plc, UK (e.g. BK300, BK357) and 3M-Neotechnic Ltd, UK (e.g. Spraymiser™).

In various embodiments, the MDIs may also be used in conjunction with other structures such as, without limitation, overwrap packages for storing and containing the MDIs, including those described in U.S. Pat. Nos. 6,119,853; 6,179,118; 6,315,112; 6,352,152; 6,390,291; and 6,679,374, as well as dose counter units such as, but not limited to, those described in U.S. Pat. Nos. 6,360,739 and 6,431,168.

Conventional bulk manufacturing methods and machinery well known to those skilled in the art of pharmaceutical aerosol manufacture may be employed for the preparation of large-scale batches for the commercial production of filled canisters. Thus, for example, in one bulk manufacturing method for preparing suspension aerosol formulations a metering valve is crimped onto an aluminium can to form an empty canister. The particulate medicament is added to a charge vessel and liquefied propellant together with the optional excipients is pressure filled through the charge vessel into a manufacturing vessel. The drug suspension is mixed before recirculation to a filling machine and an aliquot of the drug suspension is then filled through the metering valve into the canister. In one example bulk manufacturing method for preparing solution aerosol formulations, a metering valve is crimped onto an aluminium can to form an empty canister. The liquefied propellant together with the optional excipients and the dissolved medicament is pressure filled through the charge vessel into a manufacturing vessel.

In an alternative process, an aliquot of the liquefied formulation is added to an open canister under conditions which are sufficiently cold to ensure the formulation does not vaporise, and then a metering valve crimped onto the canister.

Typically, in batches prepared for pharmaceutical use, each filled canister is check-weighed, coded with a batch number and packed into a tray for storage before release testing.

Topical preparations may be administered by one or more applications per day to the affected area; over skin areas occlusive dressings may advantageously be used. Continuous or prolonged delivery may be achieved by an adhesive reservoir system.

Ointments, creams (for example an oil-in-water or water-in-oil composition such as an emulsion) and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents. Topical preparations may also optionally contain one or more solubilising agents and/or skin penetration-enhancing agents and/or surfactants and/or fragrances and/or preservatives and/or emulsifying agents.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents, suspending agents or preservatives.

In one embodiment, there is provided a pharmaceutical composition comprising a compound of formula (I) or a pharmaceutically acceptable salt thereof which is suitable for ocular administration. Such compositions may optionally contain one or more suspending agents, one or more preservatives, one or more wetting/lubricating agents and/or one or more isotonicity adjusting agents. Examples of ophthalmic wetting/lubricating agents may include cellulose derivatives, dextran 70, gelatine, liquid polyols, polyvinyl alcohol and povidone such as cellulose derivatives and polyols.

For internal administration the compounds of formula (I) and pharmaceutically acceptable salts thereof may, for example, be formulated in conventional manner for oral, nasal, parenteral or rectal administration. Formulations for oral administration include syrups, elixirs, powders, granules, tablets and capsules which typically contain conventional excipients such as binding agents, fillers, lubricants, disintegrants, wetting agents, suspending agents, emulsifying agents, preservatives, buffer salts, flavouring, colouring and/or sweetening agents as appropriate. Dosage unit forms may be preferred as described below.

The compounds of formula (I) and pharmaceutically acceptable salts thereof may in general be given by internal administration in cases wherein systemic glucocorticoid receptor agonist therapy is indicated.

Slow release or enteric coated formulations may be advantageous, particularly for the treatment of inflammatory bowel disorders.

Fluid unit dosage forms for parenteral administration may be prepared using a compound of formula (I) or a pharmaceutically acceptable salt thereof and a sterile vehicle which may be aqueous or oil based. The compound of formula (I) or a pharmaceutically acceptable salt thereof, depending on the vehicle and concentration used, may be either suspended or dissolved in the vehicle. In preparing solutions, the compound of formula (I) or a pharmaceutically acceptable salt thereof may be dissolved for injection and filter sterilised before filling into a suitable vial or ampoule and sealing. Optionally, adjuvants such as a local anaesthetic, preservatives and buffering agents may be dissolved in the vehicle. To enhance the stability, the composition may be frozen after filling into the vial and the water removed under vacuum. The lyophilised parenteral composition may be reconstituted with a suitable solvent just prior to administration. Parenteral suspensions may be prepared in substantially the same manner, except that the compound is suspended in the vehicle instead of being dissolved, and sterilisation cannot be accomplished by filtration. The compound may be sterilised by exposure to ethylene oxide before suspension in a sterile vehicle. A surfactant or wetting agent may be included in the composition to facilitate uniform distribution of the compound.

In some embodiments, the compounds of formula (I) or a pharmaceutically acceptable salt thereof will be formulated for oral administration. In other embodiments, the compounds of formula (I) or a pharmaceutically acceptable salt thereof will be formulated for inhaled administration. In further embodiments, the compounds of formula (I) or a pharmaceutically acceptable salt thereof may be formulated for intranasal administration.

The compounds and pharmaceutical formulations according to the invention may be used in combination with or include one or more other therapeutic agents, for example selected from anti-inflammatory agents, anticholinergic agents (particularly an M₁/M₂/M₃ receptor antagonist), β₂₋adrenoreceptor agonists, antiinfective agents such as antibiotics or antivirals, or antihistamines. The invention thus provides, in a further aspect, a combination comprising a compound of formula (I) or a pharmaceutically acceptable salt thereof together with one or more other therapeutically active agents, for example selected from an anti-inflammatory agent such as a corticosteroid or an NSAID, an anticholinergic agent, a β₂-adrenoreceptor agonist, an antiinfective agent such as an antibiotic or an antiviral, or an antihistamine. One embodiment of the invention encompasses combinations comprising a compound of formula (I) or a pharmaceutically acceptable salt thereof together with a β₂-adrenoreceptor agonist, and/or an anticholinergic, and/or a PDE-4 inhibitor, and/or an antihistamine.

One embodiment of the invention encompasses combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

Examples of β₂-adrenoreceptor agonists include salmeterol (e.g. as the racemate or a single enantiomer, such as the R-enantiomer), salbutamol (e.g. as the racemate or a single enantiomer such as the R-enantiomer), formoterol (e.g. as the racemate or a single diastereomer such as the R,R-diastereomer), salmefamol, fenoterol, carmoterol, etanterol, naminterol, clenbuterol, pirbuterol, flerobuterol, reproterol, bambuterol, indacaterol or terbutaline and salts thereof, for example the xinafoate (1-hydroxy-2-naphthalenecarboxylate) salt of salmeterol, the sulphate salt or free base of salbutamol or the fumarate salt of formoterol. In one embodiment, the β₂₋adrenoreceptor agonists are long-acting β₂-adrenoreceptor agonists, for example compounds which provide effective bronchodilation for about 12 hours or longer.

Examples of β₂-adrenoreceptor agonists may include those described in WO02/066422A, WO02/070490, WO02/076933, WO03/024439, WO 03/072539, WO03/091204, WO04/016578, WO04/022547, WO04/037807, WO04/037773, WO04/037768, WO04/039762, WO04/039766, WO01/42193 and WO03/042160.

Examples of β₂-adrenoreceptor agonists include:

3-(4-{[6-({(2R)-2-hydroxy-2-[4-hydroxy-3-(hydroxymethyl)phenyl]ethyl}amino)hexyl]oxy}butyl)benzenesulfonamide;

3-(3-{[7-({(2R)-2-hydroxy-2-[4-hydroxy-3-hydroxymethyl)phenyl]ethyl}-amino)heptyl]oxy}propyl)benzenesulfonamide;

4-{(1R)-2-[(6-{2-[(2,6-dichlorobenzyl)oxy]ethoxy}hexyl)amino]-1-hydroxyethyl}-2-(hydroxymethyl)phenol;

4-{(1R)-2-[(6-{4-[3-(cyclopentylsulfonyl)phenyl]butoxy}hexyl)amino]-1-hydroxyethyl}-2-(hydroxymethyl)phenol;

N-[2-hydroxyl-5-[(1R)-1-hydroxy-2-[[2-4-[[(2R)-2-hydroxy-2-phenylethyl]amino]phenyl]ethyl]amino]ethyl]phenyl]foramide,

N-2{2-[4-(3-phenyl-4-methoxyphenyl)aminophenyl]ethyl}-2-hydroxy-2-(8-hydroxy-2(1H)-quinolinon-5-yl)ethylamine, and

5-[(R)-2-(2-{4-[4-(2-amino-2-methyl-propoxy)-phenylamino]-phenyl}-ethylamino)-1-hydroxy-ethyl]-8-hydroxy-1H-quinolin-2-one, and salts thereof.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

Suitable anti-inflammatory agents include corticosteroids. Examples of corticosteroids which may be used in combination with the compounds of formula (I) or a pharmaceutically acceptable salt thereof are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester(fluticasone furoate), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone esters (for example mometasone furoate), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. In one embodiment corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carboth ioic acid S-fluoromethyl ester. In one embodiment the corticosteroid is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Examples of corticosteroids may include those described in WO02/088167, WO02/100879, WO02/12265, WO02/12266, WO05/005451, WO05/005452, WO06/072599 and WO06/072600.

Non-steroidal compounds having glucocorticoid agonism that may possess selectivity for transrepression over transactivation and that may be useful in combination therapy include those covered in the following published patent applications and patents: WO03/082827, WO98/54159, WO04/005229, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651, WO03/08277, WO06/000401, WO06/000398, WO06/015870, WO06/108699, WO07/000334 and WO07/054294.

Examples of anti-inflammatory agents include non-steroidal anti-inflammatory drugs (NSAID's).

Examples of NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example, montelukast), iNOS inhibitors, tryptase and elastase inhibitors, beta-2 integrin antagonists and adenosine receptor agonists or antagonists (for example, adenosine 2a agonists), cytokine antagonists (for example, chemokine antagonists, such as a CCR3 antagonist) or inhibitors of cytokine synthesis, or 5-lipoxygenase inhibitors. An iNOS (inducible nitric oxide synthase inhibitor) is preferably for oral administration. Suitable iNOS inhibitors include those disclosed in WO93/13055, WO98/30537, WO02/50021, WO95/34534 and WO99/62875. Suitable CCR3 inhibitors include those disclosed in WO02/26722.

In one embodiment, the invention provides the use of the compounds of formula (I) or a pharmaceutically acceptable salt thereof in combination with a phosphodiesterase 4 (PDE4) inhibitor, for example in the case of a formulation adapted for inhalation. The PDE4-specific inhibitor may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Another compound is cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep. 1996; this patent and the compounds it discloses are incorporated herein in full by reference.

Other compounds include AWD-12-281 from Elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as Cl-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther,1998, 284(1): 162), and T2585.

Further compounds are disclosed in the published international patent application WO04/024728 (Glaxo Group Ltd), PCT/EP2003/014867 (Glaxo Group Ltd) and PCT/EP2004/005494 (Glaxo Group Ltd).

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (for example, CAS 28797-61-7), darifenacin (for example, CAS 133099-04-4, or CAS 133099-07-7 for the hydrobromide sold under the name Enablex), oxybutynin (for example, CAS 5633-20-5, sold under the name Ditropan), terodiline (for example, CAS 15793-40-5), tolterodine (for example, CAS 124937-51-5, or CAS 124937-52-6 for the tartrate, sold under the name Detrol), otilonium (for example, as the bromide, CAS 26095-59-0, sold under the name Spasmomen), trospium chloride (for example, CAS 10405-02-4) and solifenacin (for example, CAS 242478-37-1, or CAS 242478-38-2, or the succinate also known as YM-905 and sold under the name Vesicare).

Other anticholinergic agents include compounds which are disclosed in U.S. patent application 60/487981, including, for example:

(3-endo)-3-(2,2-di-2-thienylethenyl)-8,8-dimethyl-8-azoniabicyclo[3.2.1]octane bromide;

(3-endo)-3-(2,2-diphenylethenyl)-8,8-dimethyl-8-azon iabicyclo[3.2.1]octane bromide;

(3-endo)-3-(2,2-diphenylethenyl)-8,8-dimethyl-8-azoniabicyclo[3.2.1]octane 4-methylbenzenesulfonate;

(3-endo)-8,8-dimethyl-3-[2-phenyl-2-(2-th ienyl)ethenyl]-8-azoniabicyclo[3.2.1]octane bromide; and/or

(3-endo)-8,8-dimethyl-3-[2-phenyl-2-(2-pyridinyl)ethenyl]-8-azoniabicyclo[3.2.1]octane bromide.

Further anticholinergic agents include compounds which are disclosedin U.S. patent application 60/511009, including, for example:

(endo)-3-(2-methoxy-2,2-di-thiophen-2-yl-ethyl)-8,8-dimethyl-8-azonia-bicyclo[3.2.1]octane iodide;

3-((endo)-8-methyl-8-aza-bicyclo[3.2.1 ]oct-3-yl)-2,2-diphenyl-propionitrile;

(endo)-8-methyl-3-(2,2,2-triphenyl-ethyl)-8-aza-bicyclo[3.2.1]octane;

3-((endo)-8-methyl-8-aza-bicyclo[3.2.1]oct-3-yl)-2,2-diphenyl-propionamide;

3-((endo)-8-methyl-8-aza-bicyclo[3.2.1]oct-3-yl)-2,2-diphenyl-propionic acid;

(endo)-3-(2-cyano-2,2-diphenyl-ethyl)-8,8-dimethyl-8-azonia-bicyclo[3.2.1]octane iodide;

(endo)-3-(2-cyano-2,2-diphenyl-ethyl)-8,8-dimethyl-8-azonia-bicyclo[3.2.1]octane bromide;

3-((endo)-8-methyl-8-aza-bicyclo[3.2.1]oct-3-yl)-2,2-diphenyl-propan-1-ol;

N-benzyl-3-((endo)-8-methyl-8-aza-bicyclo[3.2.1]oct-3-yl)-2,2-diphenyl-propionamide;

(endo)-3-(2-carbamoyl-2,2-diphenyl-ethyl)-8,8-dimethyl-8-azonia-bicyclo[3.2.1]octane iodide;

1-benzyl-3-[3-((endo)-8-methyl-8-aza-bicyclo[3.2.1]oct-3-yl)-2,2-diphenyl-propyl]-urea;

1-ethyl-3-[3-((endo)-8-methyl-8-aza-bicyclo[3.2.1]oct-3-yl)-2,2-diphenyl-propyl]-urea;

N-[3-((endo)-8-methyl-8-aza-bicyclo[3.2.1]oct-3-yl)-2,2-diphenyl-propyl]-acetamide;

N-[3-((endo)-8-methyl-8-aza-bicyclo[3.2.1]oct-3-yl)-2,2-diphenyl-propyl]-benzamide;

3-((endo)-8-methyl-8-aza-bicyclo[3.2.1]oct-3-yl)-2,2-di-thiophen-2-yl-propionitrile;

(endo)-3-(2-cyano-2,2-di-thiophen-2-yl-ethyl)-8,8-dimethyl-8-azonia-bicyclo[3.2.1]octane iodide;

N-[3-((endo)-8-methyl-8-aza-bicyclo[3.2.1]oct-3-yl)-2,2-diphenyl-propyl]-benzenesulfonamide;

[3-((endo)-8-methyl-8-aza-bicyclo[3.2.1]oct-3-yl)-2,2-diphenyl-propyl]-urea;

N-[3-((endo)-8-methyl-8-aza-bicyclo[3.2.1]oct-3-yl)-2,2-diphenyl-propyl]-methanesulfonamide; and/or

(endo)-3-{2,2-diphenyl-3-[(1-phenyl-methanoyl)-amino]-propyl}-8,8-dimethyl-8-azonia-bicyclo[3.2.1]octane bromide.

Further compounds include:

(endo)-3-(2-methoxy-2,2-di-thiophen-2-yl-ethyl)-8 ,8-dimethyl-8-azonia-bicyclo[3.2.1]octane iodide;

(endo)-3-(2-cyano-2,2-diphenyl-ethyl)-8,8-dimethyl-8-azonia-bicyclo[3.2.1]octane iodide;

(endo)-3-(2-cyano-2,2-diphenyl-ethyl)-8,8-dimethyl-8-azonia-bicyclo[3.2.1]octane bromide;

(endo)-3-(2-carbamoyl-2,2-diphenyl-ethyl)-8,8-dimethyl-8-azonia-bicyclo[3.2.1]octane iodide;

(endo)-3-(2-cyano-2,2-di-thiophen-2-yl-ethyl)-8,8-dimethyl-8-azonia-bicyclo[3.2.1]octane iodide; and/or

(endo)-3-{2,2-diphenyl-3-[(1-phenyl-methanoyl)-amino]-propyl}-8,8-dimethyl-8-azonia-bicyclo[3.2.1]octane bromide.

Examples of antihistamines (also referred to as H1-receptor antagonists) include any one or more of the numerous antagonists known which inhibit H1-receptors, and are safe for human use. First generation antagonists, include derivatives of ethanolamines, ethylenediamines, and alkylamines, such as diphenylhydramine, pyrilamine, clemastine, chlorpheniramine. Second generation antagonists, which are non-sedating, include loratidine, desloratidine, terfenadine, astemizole, acrivastine, azelastine, levocetirizine fexofenadine and cetirizine.

Examples of anti-histamines include loratidine, desloratidine, fexofenadine, cetirizine, levocabastine, olopatadine, amlexanox and epinastine.

In one embodiment the invention provides a combination comprising a compound of formula (I), or a pharmaceutically acceptable salt thereof, together with an H1 antagonist. Examples of H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In a further embodiment the invention provides a combination comprising a compound of formula (I), or a pharmaceutically acceptable salt thereof, together with an H3 antagonist (and/or inverse agonist). Examples of H3 antagonists include, for example, those compounds disclosedin WO2004/035556 andin WO2006/045416. Other histamine receptor antagonists which may be usedin combination with the compounds of formula (I), or a pharmaceutically acceptable salt thereof, include antagonists (and/or inverse agonists) of the H4 receptor, for example, the compounds disclosedin Jablonowski et al., J. Med. Chem. 46:3957-3960 (2003).

The invention thus provides, in a further aspect, a combination comprising a compound of formula (I) or a pharmaceutically acceptable salt thereof together with a PDE4 inhibitor.

The invention thus provides, in a further aspect, a combination comprising a compound of formula (I) or a pharmaceutically acceptable salt thereof together with a β₂-adrenoreceptor agonist.

The invention thus provides, in a further aspect, a combination comprising a compound of formula (I) or a pharmaceutically acceptable salt thereof together with a corticosteroid.

The invention thus provides, in a further aspect, a combination comprising a compound of formula (I) or a pharmaceutically acceptable salt thereof together with another non-steroidal GR agonist.

The invention thus provides, in a further aspect, a combination comprising a compound of formula (I) or a pharmaceutically acceptable salt thereof together with an anticholinergic.

The invention thus provides, in a further aspect, a combination comprising a compound of formula (I) or a pharmaceutically acceptable salt thereof together with an antihistamine.

The invention thus provides, in a further aspect, a combination comprising a compound of formula (I) and/or a pharmaceutically acceptable salt thereof together with a PDE4 inhibitor and a β₂-adrenoreceptor agonist.

The invention thus provides, in a further aspect, a combination comprising a compound of formula (I) and/or a pharmaceutically acceptable salt thereof together with an anticholinergic and a PDE-4 inhibitor.

The individual compounds of such combinations may be administered either sequentially or simultaneously in separate or combined pharmaceutical formulations. In one embodiment, the individual compounds will be administered simultaneously in a combined pharmaceutical formulation. Appropriate doses of known therapeutic agents will readily be appreciated by those skilledin the art.

The combinations referred to above may conveniently be presented for use in the form of a pharmaceutical formulation and thus pharmaceutical formulations comprising a combination as defined above together with a pharmaceutically acceptable diluent or carrier represent a further aspect of the invention.

The invention thus provides, in a further aspect, a pharmaceutical composition comprising a combination of a compound of formula (I) or a pharmaceutically acceptable salt thereof together with another therapeutically active agent.

The invention thus provides, in a further aspect, a pharmaceutical composition comprising a combination of a compound of formula (I) or a pharmaceutically acceptable salt thereof together with a PDE4 inhibitor.

The invention thus provides, in a further aspect, a pharmaceutical composition comprising a combination of a compound of formula (1) or a pharmaceutically acceptable salt thereof together with a β₂-adrenoreceptor agonist.

The invention thus provides, in a further aspect, a pharmaceutical composition comprising a combination of a compound of formula (I) or a pharmaceutically acceptable salt thereof together with a corticosteroid.

The invention thus provides, in a further aspect, a pharmaceutical composition comprising a combination of a compound of formula (I) or a pharmaceutically acceptable salt thereof together with another non-steroidal GR agonist.

The invention thus provides, in a further aspect, a pharmaceutical composition comprising a combination of a compound of formula (I) or a pharmaceutically acceptable salt thereof together with an anticholinergic.

The invention thus provides, in a further aspect, a pharmaceutical composition comprising a combination of a compound of formula (I) or a pharmaceutically acceptable salt thereof together with an antihistamine.

The invention thus provides, in a further aspect, a pharmaceutical composition comprising a combination of a compound of formula (I) or a pharmaceutically acceptable salt thereof together with a PDE4 inhibitor and a β₂-adrenoreceptor agonist.

The invention thus provides, in a further aspect, a pharmaceutical composition comprising a combination of a compound of formula (I) or a pharmaceutically acceptable salt thereof together with an anticholinergic and a PDE-4 inhibitor.

A process according to the invention for the preparation of compounds of formula (I) comprises coupling of a carboxylic acid of formula (II):

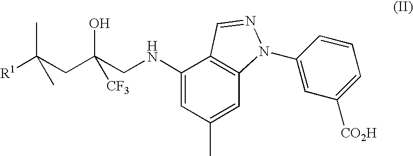

wherein R¹ is as defined above for compounds of formula (I) with an amine HNR³R⁴;

wherein R³ and R⁴ are as defined above for compounds of formula (I).

This coupling may be conducted, for example, using HATU (O-(7-azabenzotriazol-1-yl)-N,N,N′,N′-tetramethyluronium hexafluorophosphate) in the presence of a suitable base such as N,N-diisopropylethylamine in a suitable solvent such as DMF. The coupling may also be conducted using alternative, conventional conditions for amide bond formation known in the art.

Alternatively certain compounds of formula (I) may be prepared from the carboxylic acid (II) by two sequential amide couplings firstly with an amino acid followed by a second coupling with ammonia, for example sequential couplings of the carboxylic acid (II) with 4-hydroxyproline and then ammonia would provide compound (I) in which R² represents

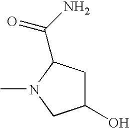

The carboxylic acid (II) may be obtained by deprotection of a suitable protected derivative (III)

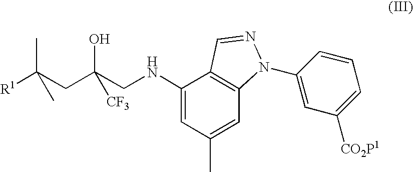

wherein R¹ is as defined above for compounds of formula (I) and P¹ represents a suitable ester protecting group, for example a benzyl ester. In the case of the benzyl protecting group, deprotection may be conveniently conducted by hydrogenolysis over palladium on carbon in ethanol. Alternative protecting groups suitable for use according to the present invention are well known to those skilledin the art and may be usedin a conventional manner. See, for example, “Protective groups in organic synthesis” by T. W. Greene and P. G. M. Wuts (John Wiley & sons 1999) or “Protecting Groups” by P. J. Kocienski (Georg Thieme Verlag 1994).

Intermediates of formula (III) may be obtained by reaction of an epoxide of formula (IV):

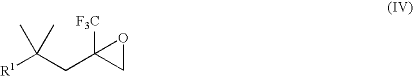

wherein R¹ is as defined above for compounds of formula (I), with a 4-amino-1-arylindazole of formula (V):

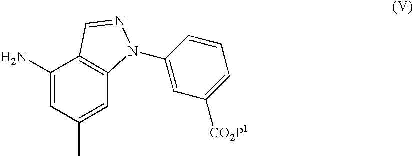

wherein P¹ is an ester protecting group as defined above for compounds of formula (III).

The epoxide opening reaction may be performed, for example, by heating the epoxide (IV) and aminoindazole (V) in acetonitrile solution at 85° C. in the presence of ytterbium(III) triflate as catalyst (Synthetic Communications 2003, 33, 2989-2994 and Bioorg. Med. Chem. Letters. 2001, 11, 1625-1628).

Compound of formula (IV) wherein R¹ represent 5-fluoro-2-methoxy-phenyl is described in racemic form in WO04/063163 and has also been described as separate enantiomers in WO05/234250, WO05/040145 and in Bioorg. Med. Chem. Letters. 2006, 16, 654-657.

Compounds of formula (V) are novel and form another aspect of the invention and may be prepared by reaction of 6-methyl-1H-indazol-4-amine (VI):

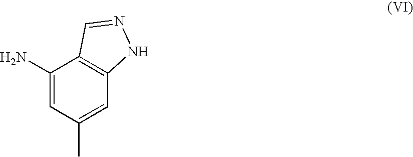

with aryl iodides of formula (VII)

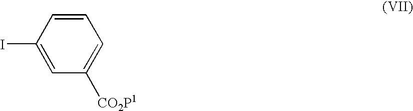

wherein P¹ is an ester protecting group as defined for compounds of formula (III).

The reaction of (VI) with (VII) may be performed in the presence of a copper(I) catalyst, such as copper(I) iodide and a weak base such as potassium carbonate or potassium phosphate and an amine ligand such as L-proline, cyclohexanediamine, N,N′-dimethylcyclohexanediamine or N,N′-dimethylethylenediamine in a variety of solvents including toluene, dioxane, N,N-dimethylformamide, N,N-dimethylacetamide and dimethylsulfoxide at a temperature in the range 60-160° C., most typically 110° C. Representative procedures are reportedin the literature: Synthesis 2005, 3, 496-499, J. Org. Chem., 2004, 69, 5578-5587 and J. Am. Chem. Soc., 2001, 123, 7727-7729.

Alternatively, compounds of formula (V) may be prepared by similar reaction of 6-methyl-4-nitro-1H-indazole (VIII)

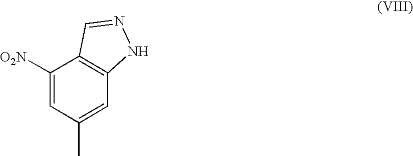

with the aryl iodides (VII) followed by reduction of the nitro group by, for example, hydrogenation over palladium on carbon.

An alternative process according to the invention for the preparation of compounds of formula (III) comprises reaction of an amine of formula (IX):

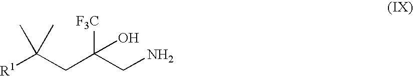

wherein R¹ is as defined above for compounds of formula (I) with a 4-bromo-1-arylindazole of formula (X):

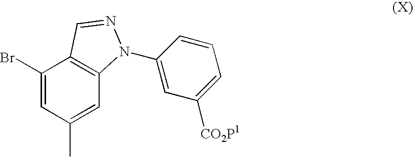

wherein P¹ is an ester protecting group as defined above for compounds of formula (III).

This coupling reaction may be conveniently carried out using palladium catalysis of the type described by Buchwaldin Topics in Current Chemistry, 2002, 219, 131-209. For example, the coupling reaction may be conducted using tris(dibenzylideneacetone)dipalladium(0), racemic BINAP (2,2′-bis(diphenylphosphino)-1,1′-binaphthyl) and sodium tert-butoxide in toluene at reflux temperature or using microwave heating.

The compound of formula (IX) wherein R¹ represents 5-fluoro-2-methoxy-phenyl is known in racemic form (WO 05/003098, WO 03/082827). Compounds of formula (IX) may also be prepared by opening epoxides of formula (IV) with benzylamine followed by removal of the benzyl group by hydrogenolysis using, for example, palladium on carbon as catalyst.

Individual enantiomers of compounds of formula (IX) may be obtained, for example, by separation by HPLC on a chiral column of the racemic material (IX) or a protected version (Xl) thereof;

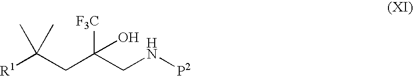

wherein the group R¹ is as defined above for compounds of formula (I), and P² represents a protecting group which is removed following enantiomer separation.

In one embodiment, P² represents a benzyloxycarbonyl (CBZ), or benzyl protecting group. However, those skilledin the art could envisage the use of other protecting groups as alternatives. The CBZ or benzyl protecting groups may be removed by, for example, hydrogenolysis over a suitable catalyst such as palladium on carbon.

Where this protecting group P² in compound (XI) contains an additional chiral centre of defined stereochemistry, for example, in the (R)-1-phenylethylamine derivative (XII)

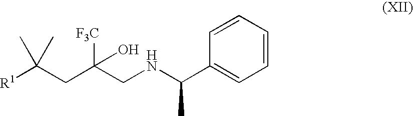

wherein the group R¹ are as defined above for compounds of formula (I), the resulting diastereoisomers may be separated by chromatography on a non-chiral or chiral support. As before, deprotection by hydrogenolysis following isomer separation provides the single enantiomers of compound (IX).

Compounds of formula (XI) may be prepared directly by protection of the racemic amine (IX). Alternatively intermediates of formula (XI) and (XII) may be prepared by the reaction of the epoxide (IV) with an amine P²—NH₂.

The epoxide opening reaction may be performed, for example, by heating with the amine in ethanol solution at 50-80° C.

Compounds of formula (X) are novel and form another aspect of the invention and may be prepared by cyclisation of a hydrazone of formula (XIII)

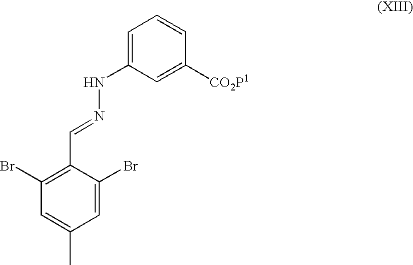

wherein P¹ is an ester protecting group as defined for compounds of formula (III).

Alternatively, compounds of formula (X) may be obtained by cyclisation of the carboxylic acid (XIII, P¹═H) followed by ester protection.

This intramolecular N-arylation may be conducted using palladium catalysis of the type described by Buchwaldin Topics in Current Chemistry, 2002, 219, 131-209. For example, the cyclisation may be effected using tris(dibenzylideneacetone)dipalladium(0), racemic-BINAP (2,2′-bis(diphenylphosphino)-1,1′-binaphthyl) and tripotassium phosphate in toluene or 1,4-dioxane at reflux temperature.

Hydrazones of formula (XIII) may be prepared by reaction of the the aldehyde (XIV)

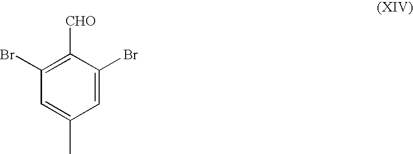

with an aryl hydrazine of formula (XV)

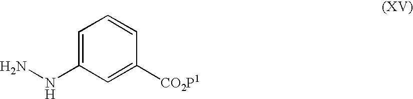

wherein P¹ is ester protecting group as defined for compounds of formula (III).

Alternatively the aldehyde (XIV) may be reacted with the unprotected carboxylic acid (XV, P¹═H) to give the hydrazone acid (XIII, P¹═H) which may either be esterified and then cyclised to give the indazole (X) or cyclised followed by ester protection to give the indazole (X).

The aldehydes (XIV) is known and may be prepared as described by Lulinski and Serwatowski in J. Org. Chem., 2003, 68, 5384-5387

Aryl hydrazines (XV) are either commercially available or may be prepared from the corresponding aniline by treatment with nitrous acid generatedin situ from sodium nitrite followed by subsequent reduction of the resulting aryldiazonium ions with tin(II) chloride according to standard literature procedures (see, for example, J Med Chem 1991, 34, 2895; J Med Chem 2000 43: 4707, J Med Chem 2003 46: 2012).

Compounds of formula (I) in which R¹ represents 5-fluoro-2-hydroxy-phenyl may be prepared by reaction of the compounds of formula (I) in which A¹ represents 5-fluoro-2-methoxy-phenyl with, for example, boron tribromide in dichloromethane solution or by treatment with lithium iodide in N-methylpyrrolidinone using microwave heating at 220 ° C.

Compounds of formula (I) may be preparedin the form of mixtures of enantiomers or diastereoisomers when mixtures of isomers are used as intermediates in the synthesis. For example, the use of a compound of formula (IV) or (IX) as a racemic mixture of enantiomers will lead to a mixture of isomers in the final product. These isomers may, if desired, be separated by conventional methods (e.g. HPLC on a chiral column).

Alternatively, separation of isomers may be performed earlier in the synthesis, for example individual isomers of compounds of formula (IV) or (IX) may be employed which may obviate the need to perform a separation of isomers as a final stage in the synthesis. The later process is, in theory, more efficient andis therefore preferred.

Compositions comprising a compound of the invention also constitute an aspect of the invention.

In addition, processes for preparing formulations including one or more compounds of formula (I) form an aspect of this invention.

Solvates of compounds of formula (I) or salts thereof, which are not physiologically acceptable may be useful as intermediates in the preparation of other compounds of formula (I) or salts thereof.

Compounds of the invention may be expected to demonstrate good anti-inflammatory properties, with predictable pharmacokinetic and pharmacodynamic behaviour. They also may be expected to have an attractive side-effect profile, demonstrated, for example, by increased selectivity for the glucocorticoid receptor over the progesterone receptor and are expected to be compatible with a convenient regime of treatment in human patients.

The invention will now be illustrated by way of the following non-limiting examples.

### EXAMPLES

### Synthetic Experimental

### ABBREVIATIONS

| | |
|---|---|
| CDCl₃ | Deuterochloroform |
| DMSO | Dimethylsulphoxide |
| EtOH | Ethanol |
| Me | Methyl |
| NMR | Nuclear magnetic resonance |
| SPE | Solid phase extraction |
| HPLC | High pressure liquid chromatography |
| LCMS | Liquid chromatography mass spectrometry |
| DMF | N,N-Dimethylformamide |
| BINAP | (2,2′-bis(diphenylphosphino)-1,1′-binaphthyl) |
| HATU | O-(7-Azabenzotriazol-1-yl)-N,N,N′,N′-tetramethyluronium |
| | hexafluorophosphate |
| HCl | Hydrochloric acid |
| RT | Room temperature |
| TFA | Trifluoroacetic acid |

Chromatographic Purification

Chromatographic purification was performed using pre-packed silica gel cartridges. The Flashmaster II is an automated multi-user flash chromatography system, available from Argonaut Technologies Ltd, which utilises disposable, normal phase, SPE cartridges (2 g to 100 g). It provides quaternary on-line solvent mixing to enable gradient methods to be run. Samples are queued using the multi-functional open access software, which manages solvents, flow-rates, gradient profile and collection conditions. The system is equipped with a Knauer variable wavelength UV-detector and two Gilson FC204 fraction-collectors enabling automated peak cutting, collection and tracking.

NMR

¹H NMR spectra were recordedin either CDCl₃ or DMSO-d₆ on either a Bruker DPX 400 or Bruker Avance DRX or Varian Unity 400 spectrometer all working at 400 MHz. The internal standard used was either tetramethylsilane or the residual protonated solvent at 7.25 ppm for CDCl₃ or 2.50 ppm for DMSO-d₆.

Mass Directed Autopreparative HPLC

System A:

Agilent 1100 series LC/MSD hardware, using electrospray positive mode (ES +ve) running chemstation 32 purification software.

Column: Zorbax Eclipse XDB-C18 prep HT (dimensions 212×100 mm, 5 μm packing), 20 ml/min solvent speed.

Aqueous solvent=Water+0.1% TFA

Organic solvent=MeCN+0.1% TFA

Gradient used:

1 min 70% Water (0.1% TFA):30% MeCN (0.1% TFA) increasing over 9 mins to 5% Water (0.1% TFA):95% MeCN (0.1% TFA) to elute compounds.

System B:

Purifications were carried out using a Micromass ZQ plafform. The column was a 100 mm×20 mm Supelco LCABZ++ with stationary phase particle size of 5 μm.

Solvents: A:water+0.1% formic acid
B: MeCN:water 95:5+0.05% formic acid

Gradient 50-90% B over 10 minutes

Flow rate 20 mL/min

LCMS System

The LCMS system used was as follows:

System A:
Column: 3.3 cm×4.6 mm ID, 3 μm ABZ+PLUS from Supelco
Flow Rate: 3 ml/min
Injection Volume: 5 μl
Temp: RT
UV Detection Range: 215 to 330 nm

Solvents: A:0.1% Formic Acid+10 mMolar Ammonium Acetate.
B:95% Acetonitrile+0.05% Formic Acid

Gradient:

| Time | A % | B % |
|---|---|---|
| 0.00 | 100 | 0 |
| 0.70 | 100 | 0 |
| 4.20 | 0 | 100 |
| 5.30 | 0 | 100 |
| 5.50 | 100 | 0 |

System B:
Column: 50 mm×2.1 mm ID, 1.7 μm Acquity UPLC BEH C₁₈
Flow Rate: 1 mL/min
Injection Volume: 0.5 μL
Temp: 40° C.
UV Detection Range: 220 to 330 nm

Solvents: A:0.1% Formic Acid+10 mM Ammonium Acetate.
B:95% Acetonitrile+0.05% Formic Acid

Gradient:

| Time (min) | A % | B % |
|---|---|---|
| 0 | 97 | 3 |
| 0.1 | 97 | 3 |
| 1.4 | 0 | 100 |
| 1.9 | 0 | 100 |
| 2 | 97 | 3 |

### Intermediate 1: Phenylymethyl 3-(4-amino-6-methyl-1H-indazol-1-yl)benzoate

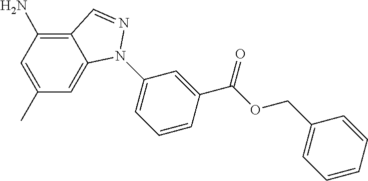

6-Methyl-1H-indazol-4-amine hydrochloride (0.5 g, 2.7 mmol), phenylmethyl 3-iodobenzoate (0.9 g, 2.6 mmol), copper (I) iodide (14 mg, 0.07 mmol), potassium carbonate (1.2 g, 8.68 mmol) and trans-N,N′-dimethyl-1,2-cyclohexanediamine (20 mg, 0.14 mmol) were heated together in DMF (5 mL) at reflux overnight. The mixture was cooled, poured into a mixture of water and ethyl acetate and filtered through celite. The organic phase was separated, combined with a second ethyl acetate extract, washed successively with water and brine and then dried over anhydrous sodium sulphate and evaporated under reduced pressure. The residue was purified by silica gel column chromatography (75 g) eluting with 1 to 5% gradient of ethyl acetate in dichloromethane to give the title compound (0.3 g).

¹H-NMR: (CDCl₃, 400 MHz) δ 8.46 (t, 1H), 8.10 (s, 1H), 8.06 (m, 1H), 7.96 (m, 1H), 7.61 (t, 1H), 7.49 (m, 2H), 7.42 (m, 2H), 7.38 (m, 1H), 6.96 (s, 1H), 6.31 (s, 1H), 5.44 (s, 2H), 4.15 (m, 2H), 2.42 (s, 3H)

### Intermediate 2: Phenylmethyl 3-(4-{[4-[5-fluoro-2-(methyloxy)phenyl]-2-hydroxy-4-methyl-2-(trifluoromethyl)pentyl]amino}-6-methyl-1H-indazol-1-yl)benzoate

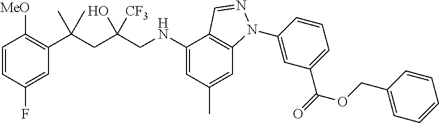

A mixture of racemic 2-{2-[5-fluoro-2-(methyloxy)phenyl]-2-methylpropyl}-2-(trifluoromethyl)oxirane (which may be prepared according to WO 04/063163, 350 mg, 1.2 mmol), phenylmethyl 3-(4-amino-6-methyl-1H-indazol-1-yl)benzoate (357 mg, 1.0 mmol) and ytterbium(III) triflate (124 mg, 0.2 mmol) in acetonitrile (2 mL) was heated at 85° C. for 18 hours when the temperature was raised to reflux temperature and heating continued for a further 21 hours. The mixture was cooled to room temperature and partitioned between dichloromethane (50 mL) and aqueous sodium bicarbonate (50 mL). The aqueous layer was extracted again with dichloromethane (50 mL) and the combined organic extracts were dried over anhydrous sodium sulphate and evaporated. The residue was purified by silica gel chromatography using the Flashmaster II (50 g cartridge) eluting with a 1:1 cyclohexane:ethyl acetate gradient over 40 minutes to give the title compound as a white solid (424 mg).

¹H-NMR: (CDCl₃, 400 MHz) δ 8.40 (t, 1H), 8.04-8.07 (m, 1H), 7.97 (s, 1H), 7.91 (ddd, 1H), 7.60 (t, 1H), 7.46-7.49 (m, 2H), 7.35-7.43 (m, 4H), 7.17 (dd, 1H), 6.99 (m, 2H), 6.85 (dd, 1H), 5.70 (broad s, 1H), 5.42 (s, 2H), 3.87 (s, 3H), 3.35 (d, 1H), 3.12 (d, 1H), 2.88 (d, 1H), 2.38 (s, 3H), 2.28 (d, 1H), 1.46 (s, 3H), 1.43 (s, 3H)

### Intermediate 3:3-[4-({4-[5-Fluoro-2-(methyloxy)phenyl]-2-hydroxy-2,4-dimethylpentyl}amino)-6-methyl-1H-indazol-1-yl]benzoic acid3-(4-{[4-[5-Fluoro-2-(methyloxy)phenyl]-2-hydroxy-4-methyl-2-(trifluoromethyl)pentyl]amino}-6-methyl-1H-indazol-1-yl)benzoic acid

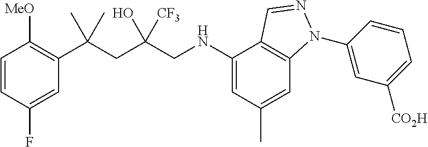

Phenylmethyl 3-(4-{[4-[5-fluoro-2-(methyloxy)phenyl]-2-hydroxy-4-methyl-2-(trifluoromethyl)pentyl]amino}-6-methyl-1H-indazol-1-yl)benzoate (2.33 g, 3.59mmol) was suspendedin ethanol (75 mL) and hydrogenated at 5 atmospheres at room temperature in the presence of 10% palladium on carbon (700 mg) for 16 hours. The mixture was filtered through Celite and the filtrate evaporated to provide the title compound (1.85 g).

LCMS (System A): t_{RET}=4.06 min; MH⁺=560

### Intermediate 4: (4S)-1-{[3-(4-{[4-[5-Fluoro-2-(methyloxy)phenyl]-2-hydroxy-4-methyl-2-(trifluoromethyl)pentyl]amino}-6-methyl-1H-indazol-1-yl)phenyl]carbonyl}-4-hydroxy-D-proline

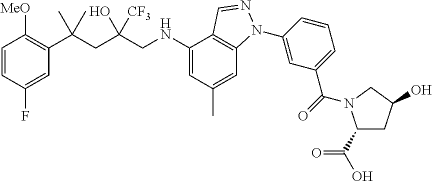

N,N-Diisopropylethylamine (0.087 mL, 0.5 mmol) and HATU (40 mg, 0.105 mmol) was added to a solution of 3-[4-({4-[5-fluoro-2-(methyloxy)phenyl]-2-hydroxy-2,4-dimethylpentyl}amino)-6-methyl-1H-indazol-1-yl]benzoic acid (56 mg, 0.1 mmol) in DMF (1 mL) and the mixture stirred for 5 min. (4S)-4-Hydroxy-D-proline (26 mg, 0.2 mmol) was added and the mixture stirred at room temperature for 48 hr. The DMF was evaporated under a stream of nitrogen overnight and the residue was dissolvedin a 1:1 mixture of methanol and DMSO containing a drop of dilute hydrochloric acid and the solution purified by mass directed autopreparation (System B). Product containing fractions were partitioned between water and dichloromethane (50 mL of each) and the organic phase was separated, dried over anhydrous sodium sulphate and evaporated to give the title compound (20.7 mg).

LCMS (System A): t_{RET}=3.54 min; MH⁺=673

### Intermediate 5: (4R)-1-{[3-(4-{[4-[5-Fluoro-2-(methyloxy)phenyl]-2-hydroxy-4-methyl-2-(trifluoromethyl)pentyl]amino}-6-methyl-1H-indazol-1-yl)phenyl]carbonyl}-4-hydroxy-D-proline

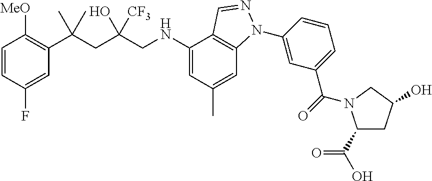

Prepared similarly to Intermediate 4 from 3-[4-({4-[5-fluoro-2-(methyloxy)phenyl]-2-hydroxy-2,4-dimethylpentyl}amino)-6-methyl-1H-indazol-1-yl]benzoic acid and (4R)-4-hydroxy-D-proline.

LCMS (System A): t_{RET}=3.63 min; MH⁺=673

### Intermediate 6: 1-({[3-(4-{[4-[5-Fluoro-2-(methyloxy)phenyl]-2-hydroxy-4-methyl-2-(trifluoromethyl)pentyl]amino}-6-methyl-1H-indazol-1-yl)phenyl]carbonyl}amino)cyclopropanecarboxylic acid

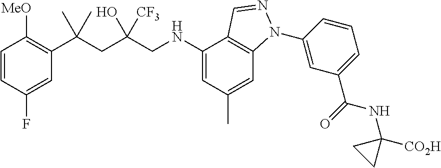

N,N-Diisopropylethylamine (0.101 mL) and HATU (46.3 mg) were added to a solution of 3-[4-({4-[5-fluoro-2-(methyloxy)phenyl]-2-hydroxy-2,4-dimethylpentyl-amino)-6-methyl-1H-indazol-1-yl]benzoic acid (65 mg, 0.116 mmol) in DMF (2.1 mL) and the mixture stirred under nitrogen for 10 min. 1-Aminocyclopropanecarboxylic acid (29.3 mg) was added and the mixture stirred overnight. The resulting suspension was diluted with methanol, filtered and purified by mass directed autopreparation (System B). Product containing fractions were combined and partitioned between dichloromethane and saturated aqueous sodium bicarbonate. The aqueous phase was adjusted to pH1 with 2M hydrochloric acid and extracted with dichloromethane (×3) and the combined organic extracts were washed successively with water and brine, dried through a hydrophobic frit and evaporated to give the title compound (8.1 mg).

LCMS (System A): t_{RET}=3.69 min; MH⁺=643

Washing of the initial dichloromethane layer with water and brine, followed by drying through a hydrophobic frit and evaporation provided a further 28.2 mg of the title compound.

### Intermediate 7: 1-({[3-(4-{[4-[5-Fluoro-2-(methyloxy)phenyl]-2-hydroxy-2-(trifluoromethyl)pentyl]amino}-6-methyl-1H-indazol-1-yl)phenyl]carbonyl}amino)cyclobutanecarboxylic acid

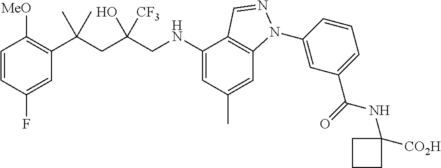

1-Chloro-N,N,2-trimethyl-1-propen-1-amine (11.94 mg, 0.0894 mmol) was added to a solution of 3-[4-({4-[5-fluoro-2-(methyloxy)phenyl]-2-hydroxy-2,4-dimethylpentyl}amino)-6-methyl-1H-indazol-1-yl]benzoic acid (50 mg, 0.0894 mmol) in anhydrous chloroform (0.1 mL) and the mixture stirred under nitrogen at room temperature for 5 min. A solution of 1-aminocyclobutanecarboxylic acid (15.4 mg) and N,N-diisopropylethylamine (0.0233 mL) in anhydrous chloroform (0.1 mL) was added and the mixture stirred under nitrogen at room temperature for 2.5 hours. The chloroform was removed with a stream of nitrogen and the residue was dissolvedin a mixture of DMSO:methanol (1:1) and purified by mass directed autopreparation (System B). Product containing fractions were combined and partitioned between dichloromethane and water. The aqueous phase was re-extracted with dichloromethane and the combined organic extracts were washed with brine, dried through a hydrophobic frit and evaporated to give the title compound (9.4 mg). LCMS (System A): t_{RET}=3.91 min; MH⁺=657.

### Example 1

### N-Ethyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

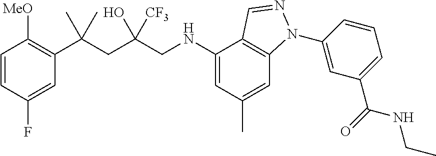

N,N-Diisopropylethylamine (0.02 mL, 0.1 mmol) was added to a solution of 3-[4-({4-[5-fluoro-2-(methyloxy)phenyl]-2-hydroxy-2,4-dimethylpentyl}amino)-6-methyl-1H-indazol-1-yl]benzoic acid (27 mg, 0.05 mmol) and HATU (19 mg, 0.05 mmol) in DMF (0.1 mL) and the mixture shaken for 1 min. This was then added to a solution of ethylamine (3.38 mg, 0.075 mmol) in DMF (0.1 mL) and the mixture stirred at room temperature overnight. The mixture was purified by mass directed autopreparation (System A) to give the title compound (9.1 mg).

LCMS (System A): t_{RET}=3.81 min; MH⁺=587

### Example 2

### 3-(4-{[4-[5-Fluoro-2-(methyloxy)phenyl]-2-hydroxy-4-methyl-2-(trifluoromethyl)pentyl]amino}-6-methyl-1H-indazol-1-yl)-N-(2-methylpropyl)benzamide

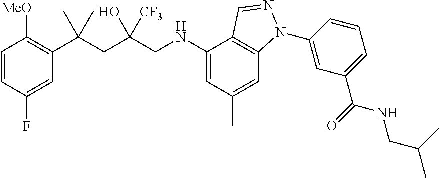

Prepared similarly to Example 1 from 3-[4-({4-[5-fluoro-2-(methyloxy)phenyl]-2-hydroxy-2,4-dimethylpentyl}amino)-6-methyl-1H-indazol-1-yl]benzoic acid and (2-methypropyl)amine.

LCMS (System A): t_{RET}=3.97 min; MH⁺=615

### Example 3

### N-Cyclopropyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

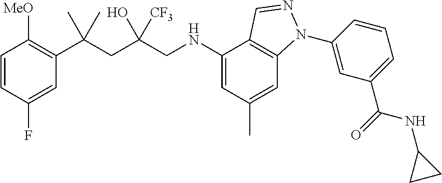

Prepared similarly to Example 1 from 3-[4-({4-[5-fluoro-2-(methyloxy)phenyl]-2-hydroxy-2,4-dimethylpentyl}amino)-6-methyl-1H-indazol-1-yl]benzoic acid and cyclopropylamine.

LCMS (System A): t_{RET}=3.81 min; MH⁺=599

### Example 4

### N-Cyclopentyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

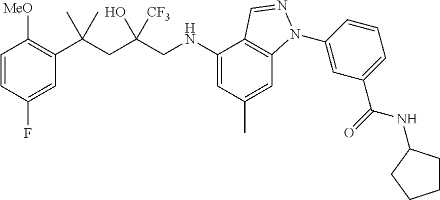

N,N-Diisopropylethylamine (0.007 mL, 0.0375 mmol) was added to a solution of 3-[4-({4-[5-fluoro-2-(methyloxy)phenyl]-2-hydroxy-2,4-dimethylpentyl}amino)-6-methyl-1H-indazol-1-yl]benzoic acid (21 mg, 0.0375 mmol) and HATU (14.3 mg, 0.0375 mmol) in DMF (0.1 mL) and the mixture added to a solution of cyclopentylamine (8.09 mg, 0.095 mmol) in DMF (0.1 mL) and stirred at room temperature overnight. DMSO (0.3 mL) was then added and the mixture purified by mass directed autopreparation (System A) to give the title compound (10.4 mg).

LCMS (System A): t_{RET}=4.04 min; MH⁺=627

### Example 5

### 3-(4-{[4-[5-Fluoro-2-(methyloxy)phenyl]-2-hydroxy-4-methyl-2-(trifluoromethyl)pentyl]amino}-6-methyl-1H-indazol-1-yl)-N-(2-hydroxyethyl)benzamide

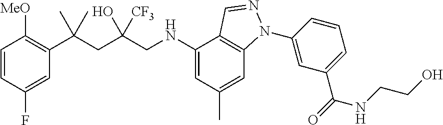

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and ethanolamine. The crude product obtained from the initial mass directed autopreparation was re-purified using the same system and the resulting product containing fractions were blown down to approximately half volume, diluted with chloroform and passed through a hydrophobic frit to give the title compound.

LCMS (System A): t_{RET}=3.57 min; MH⁺=603

### Example 6

### 3-(4-{[4-[5-Fluoro-2-(methyloxy)phenyl]-2-hydroxy-4-methyl-2-(trifluoromethyl)pentyl]amino}-6-methyl-1H-indazol-1-yl)-N-[(2S)-2-hydroxypropyl]benzamide

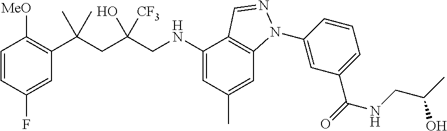

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and (2S)-1-amino-2-propanol.

LCMS (System A): t_{RET}=3.67 min; MH⁺=617

### Example 7

### 3-(4-{[4-[5-Fluoro-2-(methyloxy)phenyl]-2-hydroxy-4-methyl-2-(trifluoromethyl)pentyl]amino}-6-methyl-1H-indazol-1-yl)-N-[(2R)-2-hydroxypropyl]benzamide

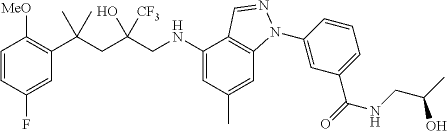

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and (2R)-1-amino-2-propanol.

LCMS (System A): t_{RET}=3.67 min; MH⁺=617

### Example 8

### 3-(4-{[4-[5-Fluoro-2-(methyloxy)phenyl]-2-hydroxy-4-methyl-2-(trifluoromethyl)pentyl]amino}-6-methyl-1H-indazol-1-yl)-N-(2-hydroxybutyl)benzamide

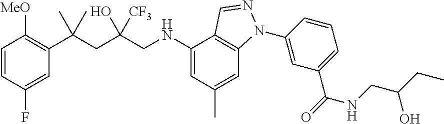

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and 1-amino-2-butanol.

LCMS (System A): t_{RET}=3.77 min; MH⁺=631

### Example 9

### N-[(2S)-2,3-dihydroxypropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

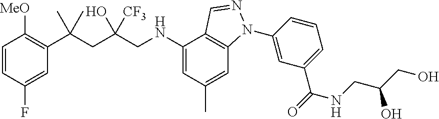

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and (2S)-3-amino-1,2-propanediol.

LCMS (System A): t_{RET}=3.51 min; MH⁺=633

### Example 10

### N-[(2R)-2,3-dihydroxypropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

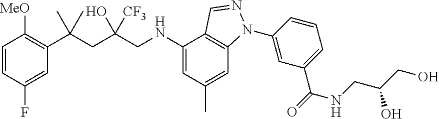

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and (2R)-3-amino-1,2-propanediol.

LCMS (System A): t_{RET}=3.51 min; MH⁺=633

### Example 11

### N-[(1S)-1-(Aminocarbonyl)propyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

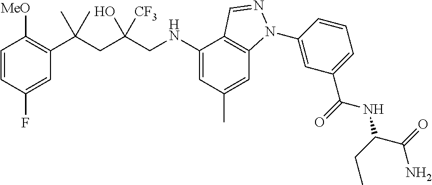

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and (2S)-2-aminobutanamide.

LCMS (System A): t_{RET}=3.61 min; MH⁺=644

### Example 12

### N-(3-Amino-3-oxopropyl)-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

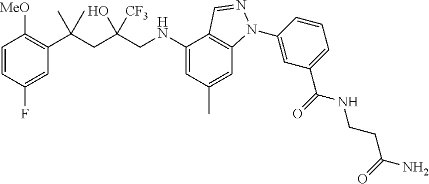

Prepared similarly to Example 1 from 3-[4-({4-[5-fluoro-2-(methyloxy)phenyl]-2-hydroxy-2,4-dimethylpentyl}amino)-6-methyl-1H-indazol-1-yl]benzoic acid and β-alaninamide.

LCMS (System A): t_{RET}=3.46 min; MH⁺=630

### Example 13

### N²-{[3-(4-{[4-[5-Fluoro-2-(methyloxy)phenyl]-2-hydroxy-4-methyl-2-(trifluoromethyl)pentyl]amino}-6-methyl-1H-indazol-1-yl)phenyl]carbonyl}-D-glutamamide

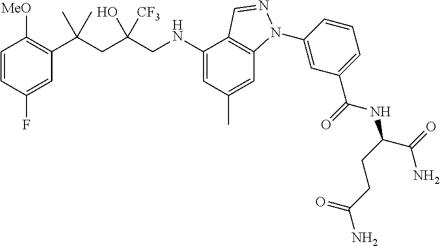

Prepared similarly to Examrple 4 from 3-[4-({4-[5-fluoro-2-(methyloxy)phenyl]-2-hydroxy-2,4-dimethylpentyl}amino)-6-methyl-1H-indazol-1-yl]benzoic acid and D-glutamamide.

LCMS (System A): t_{RET}=3.34 min; MH⁺=687

### Example 14

### N-[(1S,2R)-1-(Aminocarbonyl)-2-hydroxypropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

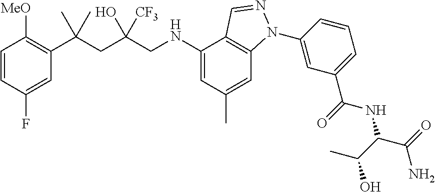

Prepared similarly to Examlple 4 from 3-[4-({4-[5-fluoro-2-(methyloxy)phenyl]-2-hydroxy-2,4-dimethylpentyl}amino)-6-methyl-1H-indazol-1-yl]benzoic acid and L-threoninamide.

LCMS (System A): t_{RET}=3.46 min; MH⁺=660

### Example 15

### N²-{[3-(4-{[4-[5-Fluoro-2-(methyloxy)phenyl]-2-hydroxy-4-methyl-2-(trifluoromethyl)pentyl]amino}-6-methyl-1H-indazol-1-yl)phenyl]carbonyl}-L-aspartamide

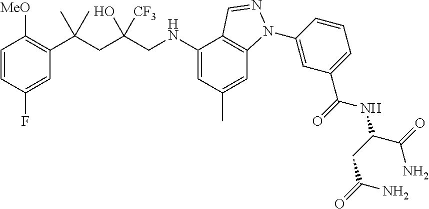

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and L-aspartamide.

LCMS (System A): t_{RET}=3.34 min; MH⁺=673

### Example 16

### N-[(1S)-1-(Aminocarbonyl)-3-(methylthio)propyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

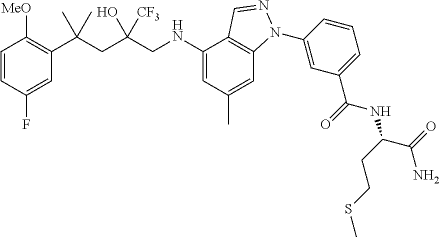

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and L-methioninamide.

LCMS (System A): t_{RET}=3.68 min; MH⁺=690

### Example 17

### N-[(1S)-1-(Aminocarbonyl)-2-methylpropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

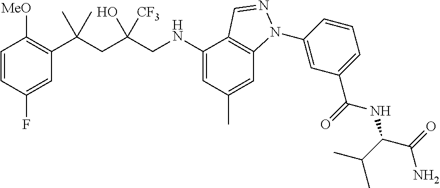

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and L-valinamide.

LCMS (System A): t_{RET}=3.68 min; MH⁺=658

### Example 18

### N-[(1R)-1-(Aminocarbonyl)-2-methylpropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

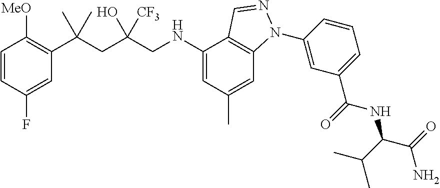

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and D-valinamide.

LCMS (System A): t_{RET}=3.68 min; MH⁺=658

### Example 19

### 1-{[3-(4-{[4-[5-Fluoro-2-(methyloxy)phenyl]-2-hydroxy-4-methyl-2-(trifluoromethyl)pentyl]amino}-6-methyl-1H-indazol-1-yl)phenyl]carbonyl}-2-piperidinecarboxamide

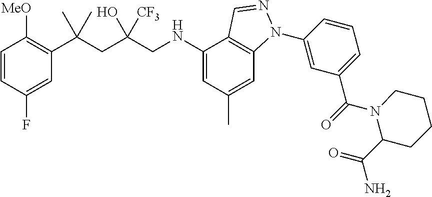

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and 2-piperidinecarboxamide.

LCMS (System A): t_{RET}=3.66 min; MH⁺=670

### Example 20

### 3-(4-{[4-[5-Fluoro-2-(methyloxy)phenyl]-2-hydroxy-4-methyl-2-(trifluoromethyl)pentyl]amino}-6-methyl-1H-indazol-1-yl)-N-[(3R)-2-oxotetrahydro-3-furanyl]benzamide

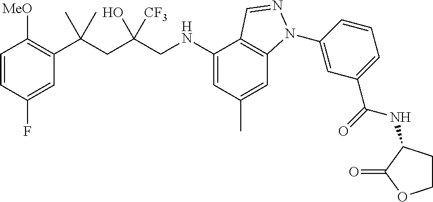

A mixture of N,N-diisopropylethylamine (0.007 mL, 0.0375 mmol), 3-[4-({4-[5-fluoro-2-(methyloxy)phenyl]-2-hydroxy-2,4-dimethylpentyl}amino)-6-methyl-1H-indazol-1-yl]benzoic acid (21 mg, 0.0375 mmol) and HATU (14.3 mg, 0.0375 mmol) in DMF (0.15 mL) was added to a solution of (3R)-3-aminodihydro-2(3H)-furanone hydrochloride (ca.18 mg, ca.0.13 mmol) in DMF (0.1 mL) containing N,N-diisopropylethylamine (0.04 mL) and the mixture allowed to stand at room temperature for 16 hours. The solvent was then removed under reduced pressure and the residue was purified by mass directed autopreparation (System A) to give the title compound.

LCMS (System A): t_{RET}=3.77 min; MH⁺=643

### Example 21

### 3-(4-{[4-[5-Fluoro-2-(methyloxy)phenyl]-2-hydroxy-4-methyl-2-(trifluoromethyl)pentyl]amino}-6-methyl-1H-indazol-1-yl)-N-[(3S)-2-oxotetrahydro-3-furanyl]benzamide

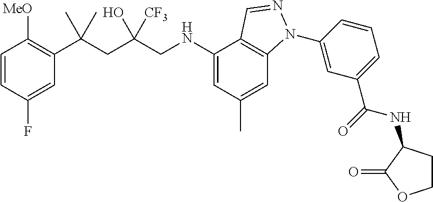

Prepared similarly to Example 20 from 3-[4-({4-[5-fluoro-2-(methyloxy)phenyl]-2-hydroxy-2,4-dimethylpentyl}amino)-6-methyl-1H-indazol-1-yl]benzoic acid and (3S)-3-aminodihydro-2(3H)-furanone hydrochloride.

LCMS (System A): t_{RET}=3.77 min; MH⁺=643

311 mg of this mixture of diastereomers was resolved by chiral HPLC on a 5 cm×20 cm Chiralpak AD column (20 μm) eluted with heptane:EtOH 2:8 with a flow rate of 75 mL/min to provide Example 21-A (diastereomer A, 124.9 mg) and Example 21-B (diastereomer B, 115.4 mg)

### Example 21-A

### (diastereomer A): Analytical chiral HPLC (25×0.46 cm Chiralpak AD column, heptane:EtOH 1:9 eluting at 1 mL/min): t_{RET}=8.7 min

LCMS: t_{RET}=3.71 min; MH⁺=643

### Example 21-B

### (diastereomer B): Analytical chiral HPLC (25×0.46 cm Chiralpak AD column, heptane:EtOH 1:9 eluting at 1 mL/min): t_{RET}=18.6 min

LCMS: t_{RET}=3.71 min; MH⁺=643

### Example 22

### 1,1,1-Trifluoro-4-[5-fluoro-2-(methyloxy)phenyl]-4-methyl-2-[({6-methyl-1-[3-({(2S)-2-[(methyloxy)methyl]-1-pyrrolidinyl]carbonyl)phenyl]-1H-indazol-4-yl}amino)methyl]-2-pentanol

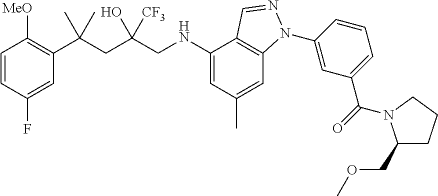

Prepared similarly to Example 1 from 3-[4-({4-[5-fluoro-2-(methyloxy)phenyl]-2-hydroxy-2,4-dimethylpentyl}amino)-6-methyl-1H-indazol-1-yl]benzoic acid and (2S)-2-[(methyloxy)methyl]pyrrolidine.

LCMS (System A): t_{RET}=3.85 min; MH⁺=657

### Example 23

### 3-(4-{[4-[5-Fluoro-2-(methyloxy)phenyl]-2-hydroxy-4-methyl-2-(trifluoromethyl)pentyl]amino}-6-methyl-1H-indazol-1-yl)-N-(3-furanylmethyl)benzamide

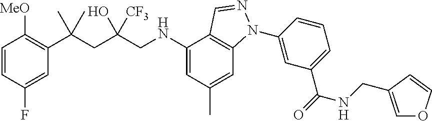

Prepared similarly to Example 1 from 3-[4-({4-[5-fluoro-2-(methyloxy)phenyl]-2-hydroxy-2,4-dimethylpentyl}amino)-6-methyl-1H-indazol-1-yl]benzoic acid and (3-furanylmethyl)amine.

LCMS (System A): t_{RET}=3.88 min; MH⁺=639

### Example 24

### N-(1,1-Dioxidotetrahydro-3-thienyl)-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

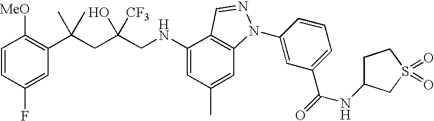

Prepared similarly to Examrple 1 from 3-[4-({4-[5-fluoro-2-(methyloxy)phenyl]-2-hydroxy-2,4-dimethylpentyl}amino)-6-methyl-1H-indazol-1-yl]benzoic acid and (1,1-dioxidotetrahydro-3-thienyl)amine.

LCMS (System A): t_{RET}=3.65 min; MH⁺=677

### Example 25

### (3R)-1-{[3-(4-{[4-[5-Fluoro-2-(methyloxy)phenyl]-2-hydroxy-4-methyl-2-(trifluoromethyl)pentyl]amino}-6-methyl-1H-indazol-1-yl)phenyl]carbonyl}-3-pyrrolidinol

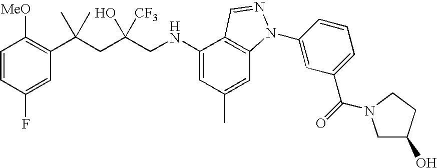

Prepared similarly to Example 20 from 3-[4-({4-[5-fluoro-2-(methyloxy)phenyl]-2-hydroxy-2,4-dimethylpentyl}amino)-6-methyl-1H-indazol-1-yl]benzoic acid and (3R)-3-pyrrolidinol.

LCMS (System A): t_{RET}=3.60 min; MH⁺=629

### Example 26

### 3-(4-{[4-[5-Fluoro-2-(methyloxy)phenyl]-2-hydroxy-4-methyl-2-(trifluoromethyl)pentyl]amino}-6-methyl-1H-indazol-1-yl)-N-(1,2,4-oxadiazol-3-ylmethyl)benzamide

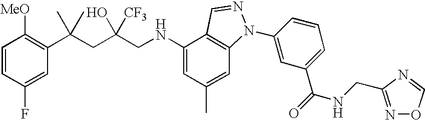

Prepared similarly to Example 5 from 3-[4-({4-[5-fluoro-2-(methyloxy)phenyl]-2-hydroxy-2,4-dimethylpentyl}amino)-6-methyl-1H-indazol-1-yl]benzoic acid and (1,2,4-oxadiazol-3-ylmethyl)amine.

LCMS (System A): t_{RET}=3.71 min; MH⁺=641

### Example 27

### 3-(4-{[4-[5-Fluoro-2-(methyloxy)phenyl]-2-hydroxy-4-methyl-2-(trifluoromethyl)pentyl]amino}-6-methyl-1H-indazol-1-yl)-N-[(3-methyl-1H-1,2,4-triazol-5-yl)methyl]benzamide

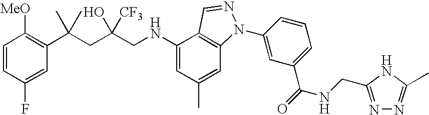

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and [(3-methyl-1H-1,2,4-triazol-5-yl)methyl]amine.

LCMS (System A): t_{RET}=3.54 min; MH⁺=654

### Example 28

### 3-(4-{[4-[5-Fluoro-2-(methyloxy)phenyl]-2-hydroxy-4-methyl-2-(trifluoromethyl)pentyl}amino)-6-methyl-1H-indazol-1-yl)benzamide

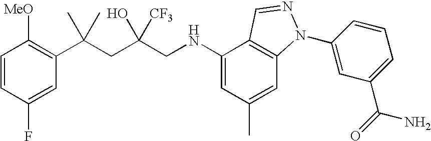

Prepared similarly to Example 4 from 3-[4-({4-[5-fluoro-2-(methyloxy)phenyl]-2-hydroxy-2,4-dimethylpentyl}amino)-6-methyl-1H-indazol-1-yl]benzoic acid and ammonia.

LCMS (System A): t_{RET}=3.68 min; MH⁺=559

### Example 29

### (4S)-1-{[3-(4-{[4-[5-Fluoro-2-(methyloxy)phenyl]-2-hydroxy-4-methyl-2-(trifluoromethyl)pentyl]amino}-6-methyl-1H-indazol-1-yl)phenyl]carbonyl}-4-hydroxy-D-prolinamide

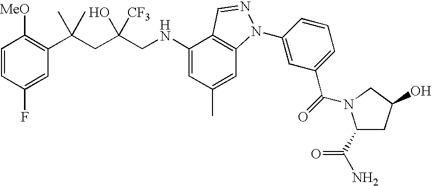

N,N-Diisopropylethylamine (0.023 mL, 0.135 mmol) was added to a solution of (4S)-1-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-4-hydroxy-D-proline (17.8 mg, 0.027 mmol) and HATU (10.6 mg, 0.028 mmol) in DMF (0.3 mL) and the mixture stirred for 10 min when ammonia in dioxan (5M, 0.53 mL, 2.65 mmol) was added. After 2 hr more ammonia in dioxan (5M, 0.2 mL, 1 mmol) was added and the mixture stirred for a further 1 hr and was then partitioned between ethyl acetate and aqueous sodium bicarbonate. The aqueous phase was re-extracted with ethyl acetate and the combined organic extracts were washed with water and brine, dried through a hydrophobic frit and evaporated. The residue (19.8 mg) was purified by mass directed autopreparation (System B). Product containing fractions were combined and partitioned between dichloromethane and saturated aqueous sodium bicarbonate. The aqueous phase was re-extracted with dichloromethane (×2) and the combined organic extracts were washed successively with water and brine, dried through a hydrophobic frit and evaporated to give the title comDound (10.7 mg).

LCMS (System A): t_{RET}=3.45 min; MH⁺=672

### Example 30

### (4R)-1-{[3-(4-{[4-[5-Fluoro-2-(methyloxy)phenyl]-2-hydroxy-4-methyl-2-(trifluoromethyl)pentyl]amino}-6-methyl-1H-indazol-1-yl)phenyl]carbonyl}-4-hydroxy-D-prolinamide

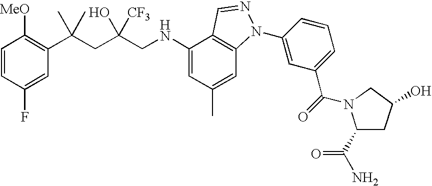

Prepared similarly to Example 29 from (4R)-1-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-4-hydroxy-D-proline and ammonia.

LCMS (System A)::t_{RET}=3.49 min; MH⁺=672

### Example 31

### 3-(4-{[4-[5-Fluoro-2-(methyloxy)phenyl]-2-hydroxy-4-methyl-2-(trifluoromethyl)pentyl]amino]-6-methyl-1H-indazol-1-yl)-N-[(3R)-1-methyl-2-oxo-3-pyrrolidinyl]benzamide

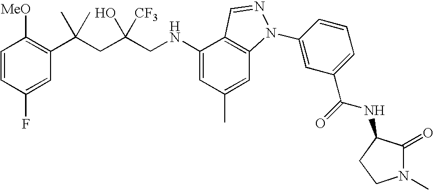

HATU (34 mg, 0.0894 mmol) was added to a solution of 3-[4-({4-[5-fluoro-2-(methyloxy)phenyl]-2-hydroxy-2,4-dimethylpentyl}amino)-6-methyl-1H-indazol-1-yl]benzoic acid (50 mg, 0.0894 mmol) in DMF (1 mL) and the mixture stirred for 10 min. (3R)-3-Amino-1-methyl-2-pyrrolidinone (10 mg, 0.0894 mmol) and N,N-diisopropylethylamine (0.031 mL, 0.179 mmol) were then added and the mixture stirred at room temperature for 6 hours. The mixture was evaporated to dryness under a stream of nitrogen and the residue purified by mass directed autopreparation (System A) to give the title compound (36 mg).

LCMS (System B): t_{RET}=1.28 min; MH⁺=656

### Example 32

### 3-(4-{[4-[5-Fluoro-2-(methyloxy)phenyl]-2-hydroxy-4-methyl-2-(trifluoromethyl)pentyl]amino}-6-methyl-1H-indazol-1-yl)-N-(5-oxo-3-pyrrolidinyl)benzamide

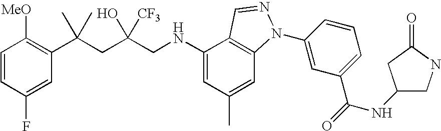

N,N-Diisopropylethylamine (0.0464 mL) and HATU (21.3 mg) were added to a solution of 3-[4-({4-[5-fluoro-2-(methyloxy)phenyl]-2-hydroxy-2,4-dimethylpentyl}amino)-6-methyl-1H-indazol-1-yl]benzoic acid (30 mg, 0.0534 mmol) in DMF (0.6 mL) and the mixture stirred under nitrogen for 10 min. 4-Amino-2-pyrrolidinone (7.29 mg) was added and the mixture stirred overnight and then diluted with methanol (1.5 mL) purified by mass directed autopreparation (System B). Product containing fractions were combined and partitioned between dichloromethane and saturated aqueous sodium bicarbonate. The aqueous phase was re-extracted with dichloromethane and the combined organic extracts were washed successively with water and brine, dried through a hydrophobic frit and evaporated to give the title compound (12.4 mg).

LCMS (System A): t_{RET}=3.52 min; MH⁺=642

### Example 33

### 4-{[3-(4-{[4-[5-Fluoro-2-(methyloxy)phenyl]-2-hydroxy-4-methyl-2-(trifluoromethyl)pentyl]amino}-6-methyl-1H-indazol-1-yl)phenyl]carbonvl}-2-piperazinone

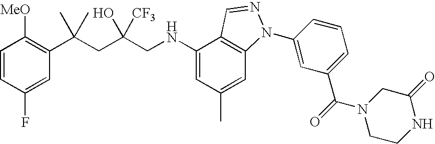

Prepared similarly to Example 32 from 3-[4-({4-[5-fluoro-2-(methyloxy)phenyl]-2-hydroxy-2,4-dimethylpentyl}amino)-6-methyl-1H-indazol-1-yl]benzoic acid and 2-piperazinone.

LCMS (System A):t_{RET}=3.47 min; MH⁺=642

### Example 34

### N-[(1R)-2-Amino-2-oxo-1-phenylethyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

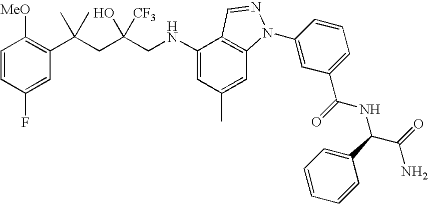

Prepared similarly to Example 32 from 3-[4-({4-[5-fluoro-2-(methyloxy)phenyl]-2-hydroxy-2,4-dimethylpentyl}amino)-6-methyl-1H-indazol-1-yl]benzoic acid and (2R)-2-amino-2-phenylethanamide.

LCMS (System A):t_{RET}=3.78 min; MH⁺=692

### Example 35

### N-[1-(Aminocarbonyl)cyclopropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

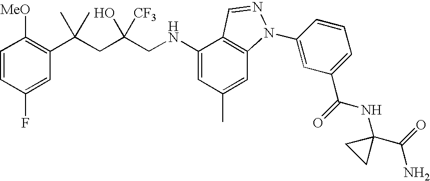

Prepared similarly to Example 32 from 1-({[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}amino)cyclopropanecarboxylic acid and ammonia.

LCMS (System A):t_{RET}=3.56 min; MH⁺=642

### Example 36

### N-[1-(Aminocarbonyl)cyclobutyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide

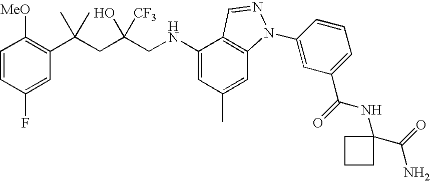

N,N-Diisopropylethylamine (0.0318 mL) and HATU (14.6 mg) were added to a solution of 1-({[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}amino)cyclobutanecarboxylic acid (24 mg, 0.0365 mmol) in anhydrous DMF (0.6 mL) and the mixture stirred under nitrogen for 10 min. Ammonia in dioxan (0.5M, 0.75 mL) was added and the mixture stirred overnight and then partitioned between dichloromethane and saturated aqueous sodium bicarbonate. The aqueous phase was re-extracted with dichloromethane and the combined organic extracts were washed successively with water, saturated aqueous citric acid, water (×2) and brine, dried through a hydrophobic frit and evaporated and the residue purified by mass directed autopreparation (System B). Product containing fractions were combined and partitioned between dichloromethane and saturated aqueous sodium bicarbonate. The aqueous phase was re-extracted with dichloromethane and the combined organic extracts were washed successively with water and brine, dried through a hydrophobic frit and evaporated to give the title comDound as a white solid (10.9 mg).

LCMS (System A): t_{RET}=3.70 min; MH⁺=656

### BIOLOGICAL EXPERIMENTAL

Glucocorticoid Receptor Binding Assay

The ability of compounds to bind to the glucocorticoid receptor was determined by assessing their ability to compete with an Alexa 555 fluorescently-labelled dexamethasone derivative. Compounds were solvated and dilutedin DMSO, and transferred directly into assay plates. Fluorescent dexamethasone and a partially purified full length glucocorticoid receptor were added to the plates, together with buffer components to stabilise the GR protein (including stabilisation peptide (Panvera catalogue number P2815)) andincubated at room temperature for 2 hours in the dark. Binding of each compound was assessed by analysing the displacement of fluorescent ligand by measuring the decrease in fluorescence polarisation signal from the mixture.

Examples 1 to 21, 21-A and 22 to 36 show glucocorticoid binding with a pIC₅₀>6.5 in this assay.

Glucocorticoid Mediated Transrepression of NFkB Activity

Human A549 lung epithelial cells were engineered to contain a secreted placental alkaline phosphatase gene under the control of the distal region of the NFkB dependent ELAM promoter as previously described in Ray, K. P., Farrow, S., Daly, M., Talabot, F. and Searle, N. “Induction of the E-selectin promoter by interleukin 1 and tumour necrosis factor alpha, andinhibition by glucocorticoids” Biochemical Journal (1997) 328: 707-15.

Compounds were solvated and dilutedin DMSO, and transferred directly into assay plates such that the final concentration of DMSO was 0.7%. Following the addition of cells (40K per well), plates were incubated for 1 hr prior to the addition of 3 ng/ml human recombinant TNFα. Following continued incubation for 16 hr, alkaline phosphatase activity was determined by measuring the change in optical density at 405 nM with time following the addition of 0.7 volumes of assay buffer (1 mg/ml p-nitrophenylphosphate dissolvedin 1M diethanolamine, 0.28M NaCl, 0.5 mM MgCl₂). Dose response curves were constructed from which EC₅₀ values were estimated.

Examples 1 to 21, 21-A and 22 to 36 show pEC₅₀>8 in this assay.

Assay for Progesterone Receptor Activity

A T225 flask of CV-1 cells at a density of 80% confluency was washed with PBS, detached from the flask using 0.25% trypsin and counted using a Sysmex KX-21N. Cells were dilutedin DMEM containing 10% Hyclone, 2 mM L-Glutamate and 1% Pen/Strep at 140 cells/μl and transduced with 10% PRb-BacMam and 10% MMTV-BacMam. 70 ml of suspension cells were dispensed to each well of white Nunc 384-well plates, containing compounds at the required concentration. After 24 h 10 μl of Steadylite were added to each well of the plates. Plates were incubatedin the dark for 10 min before reading them on a Viewlux reader. Dose response curves were constructed from which pEC₅₀ values were estimated.

Examples 2 to 34 show pEC₅₀<8 in this assay.

In describing examples according to their activity in the assays above, it will be appreciated that at least one isomer, for example, an enantiomer in a mixture of isomers (such as a racemate) has the described activity. The other enantiomer may have similar activity, less activity, no activity or may have some antagonist activity in the case of a functional assay.

Throughout the specification and the claims which follow, unless the context requires otherwise, the word ‘comprise’, and variations such as ‘comprises’ and ‘comprising’, will be understood to imply the inclusion of a statedinteger or step or group of integers but not to the exclusion of any other integer or step or group of integers or steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims.

The patents and patent applications describedin this application are herein incorporated by reference.

What is claimed is:

1. A compound of formula (I): 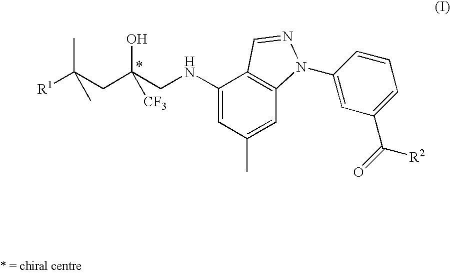
wherein
R¹ represents 5-fluoro-2-methoxy-phenyl or 5-fluoro-2-hydroxy-phenyl;
R² represents —NR³R⁴;
R³ represents hydrogen, and
R⁴ represents hydrogen, —CH₂CH₃, —CH₂CH(CH₃)₂, —CH₂CH₂OH, —CH₂CH(OH)CH₃, —CH₂CH(OH)CH₂CH₃, —CH₂CH(OH)CH₂OH, —CH(CONH₂)CH₂CH₃, —CH₂CH₂CONH₂, —CH(CONH₂)CH₂CH₂CONH₂, —CH(CONH₂)CH(OH)CH₃, —CH(CONH₂)CH₂CONH₂, —CH(CONH₂)CH(CH₃)₂, —CH(CONH₂)CH₂CH₂SCH₃, cyclopropyl, cyclopentyl, 2-oxotetrahydro-3-furanyl, 3-furanyl-methyl, 1,1-dioxidotetrahydro-3-thienyl, 1,2,4-oxadiazol-3-ylmethyl, (3-methyl-1H-1,2,4-triazol-5-yl)methyl, 1-methyl-2-oxo-3-pyrrol idinyl, 5-oxo-3-pyrrolidinyl, 2-amino-2-oxo-1-phenylethyl, 1-(aminocarbonyl)cyclopropyl or 1-(aminocarbonyl)cyclobutyl; or
R³ and R⁴ together with the nitrogen atom to which they are attached, form 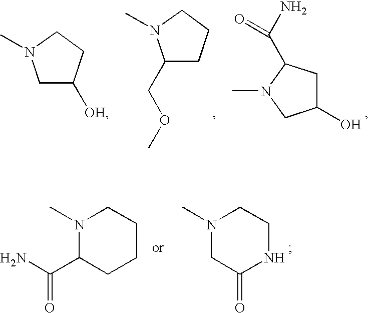
or a salt thereof.

2. A compound according to claim 1 wherein R¹ represents 5-fluoro-2-methoxy-phenyl.

3. A compound according to claim 1 wherein R³ represents hydrogen.

4. A compound according to claim 1 wherein R⁴ represents —CH₂CH₃, —CH₂CH₂OH, —CH₂CH(OH)CH₃, —CH(CONH₂)CH₂CH₃, —CH₂CH₂CONH₂, —CH(CONH₂)CH₂CH₂CONH₂, —CH(CONH₂)CH(CH₃)₂, cyclopropyl or 1,1-dioxidotetrahydro-3-thienyl.

5. A compound according to claim 4 wherein R⁴ represents —CH₂CH₃, —CH₂CH₂OH, —CH₂CH(OH)CH₃, or 1,1-dioxidotetrahydro-3-thienyl.

6. A compound which is selected from the group consisting of:
N-ethyl-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;
N-cyclopropyl-3-(4-{[4-[5-fluoro-2-(methyloxy)phenyl]-2-hydroxy-4-methyl -2-(trifluoromethyl)pentyl]amino}-6-methyl-1H-indazol-1-yl)benzamide;
3-(4-{[4-[5-fluoro-2-(methyloxy)phenyl]-2-hydroxy-4-methyl-2-(trifluoromethyl)pentyl]amino}-6-methyl-1H-indazol-1-yl)-N-(2-hydroxyethyl)benzamide;
3-(4-{[4-[5-fluoro-2-(methyloxy)phenyl]-2-hydroxy-4-methyl-2-(trifluoromethyl)pentyl]amino}-6-methyl-1H-indazol-1-yl)-N-[(2R)-2-hydroxypropyl]benzamide;
N-[(1S)-1-(aminocarbonyl)propyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;
N-(3-amino-3-oxopropyl)-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;
N²-{[3-(4-{[4-[5-fluoro-2-(methyloxy)phenyl]-2-hydroxy-4-methyl-2-(trifluoromethyl)pentyl]amino}-6-methyl-1H-indazol-1-yl)phenyl]carbonyl}-D-glutamamide;
N-[(1R)-1-(aminocarbonyl)-2-methylpropyl]-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide;
N-(1,1-dioxidotetrahydro-3-thienyl)-3-(4-{[4-[5-fluoro-2-(methyloxy)phenyl]-2-hydroxy-4-methyl-2-(trifluoromethyl)pentyl]amino}-6-methyl-1H-indazol-1-yl)benzamide; and salts thereof.

7. A compound as claimedin claim 1, or a pharmaceutically acceptable salt thereof, for use in human or veterinary medicine.

8. A compound as claimed in claim 1, or a pharmaceutically acceptable salt thereof, for use in the treatment of inflammatory and/or allergic conditions.

9-11. (canceled)

12. A method for the treatment of a human or animal subject with an inflammatory and/or allergic condition, which method comprises administering to said human or animal subject an effective amount of a compound as claimedin claim 1, or a pharmaceutically acceptable salt thereof.

13. A method for the treatment of a human or animal subject with rheumatoid arthritis, asthma, COPD, allergy and/or rhinitis, which method comprises administering to said human or animal subject an effective amount of a compound as claimedin claim 1, or a pharmaceutically acceptable salt thereof.

14. A method for the treatment of a human or animal subject with skin disease, which method comprises administering to said human or animal subject an effective amount of a compound as claimedin claim 1, or a pharmaceutically acceptable salt thereof.

15. A method for the treatment of a human or animal subject with eczema, psoriasis, allergic dermatitis, neurodermatitis, pruritis and/or hypersensitivity reactions, which method comprises administering to said human or animal subject an effective amount of a compound as claimed in claim 1, or a pharmaceutically acceptable salt thereof.
